(19) United States
(12) Patent Application Publication
(10) Pub. No.: US 2023/0380704 A1
(43) Pub. Date: Nov. 30, 2023

(54) BLOOD PRESSURE MONITORING APPARATUS

(71) Applicants: A&D COMPANY, LIMITED, Tokyo (JP); NATIONAL CEREBRAL AND CARDIOVASCULAR CENTER, Suita-shi, Osaka (JP)

(72) Inventors: Naotaka HASEBE, Kitamoto-shi (JP); Shohei MORODOME, Kitamoto-shi (JP); Masaki FURUKOSHI, Kitamoto-shi (JP); Kazunori UEMURA, Suita-shi (JP); Masaru SUGIMACHI, Suita-shi (JP); Takuya NISHIKAWA, Suita-shi (JP)

(73) Assignees: A&D COMPANY, LIMITED, Tokyo (JP); NATIONAL CEREBRAL AND CARDIOVASCULAR CENTER, Suita-shi, Osaka (JP)

(21) Appl. No.: 18/031,905

(22) PCT Filed: Oct. 11, 2021
(86) PCT No.: PCT/JP2021/037630

§ 371 Date: Apr. 14, 2023

(30) Foreign Application Priority Data
Oct. 14, 2020 (JP) 2020-173552

(51) Int. Cl. A61B 5/021; A61B 5/022

(52) U.S. Cl. CPC A61B 5/02125; A61B 5/02233; A61B 2560/0223

(57) ABSTRACT

A blood pressure monitoring apparatus including a linear relationship storage portion storing previously stored linear relationships, a blood pressure measurement portion measuring a real arterial pressure of the person to be measured, a proper relationship generation portion applying, for the person to be measured, the real arterial pressure, real compression pressures, and real pulse wave propagation velocities, to thereby generate a proper relationship on the person to be measured among the real arterial pressures of the person to be measured, the real compression pressures, and the real pulse wave propagation velocities, and a blood pressure estimation portion applying, for the person to be measured, the real compression pressures and the real pulse wave propagation velocities obtained under the real compression pressures, to the proper relationship on the person to be measured, to thereby estimate the estimated arterial pressure.

### TECHNICAL FIELD

The present invention relates to a blood pressure monitoring apparatus having a cuff wrapped around a site to be compressed that is a limb of a living body.

### BACKGROUND ART

In a commonly used indirect blood pressure measuring apparatus, blood pressure values of a person to be measured are determined based on changes of pressure pulse waves obtained as pressure oscillations of a cuff during a pressure-lowering period following the rise of the cuff compression pressure up to a compression pressure equal to or higher than the maximum blood pressure value of the person to be measured. An example thereof is the automatic blood pressure measuring apparatus described in Patent Document 1.

In the automatic blood pressure measuring apparatus described in Patent Document 1, a cuff having three inflatable bladders forming three independent air chambers is used and the maximum blood pressure value and the minimum blood pressure value are determined based on amplitude fluctuations of pulse wave signals sampled during the pressure-lowering period where the cuff compression pressure falls to a measurement end pressure value set lower than the minimum blood pressure value of a living body after rising up to a target pressure value set higher than the maximum blood pressure of the living body. Alternatively, the maximum blood pressure value is determined based on the amplitude ratio between two pulse wave signals sampled from two inflatable bladders during the pressure-lowering period, while the minimum blood pressure value is determined based on the time difference between two pulse wave signals.

### PRIOR ART DOCUMENT

### Patent Document

Patent Document 1: JP2012071059A

### SUMMARY OF THE INVENTION

### Problem to be Solved by the Invention

However, according to the above conventional blood pressure measuring apparatus, the cuff pressure is raised up to the target pressure value set higher than the maximum blood pressure value of the living body. For this reason, the cuff pressure is raised until the blood flow stops at the artery of the living body limb around which the cuff is wrapped, resulting in a drawback that the living body may feel insecure about how hard the limb is compressed or that the living body may feel a heavy burden. For example, since the cuff tightening force is increased until the blood flow stops at the artery of living body limb, the living body may feel insecure and become psychologically unstable during the measurement, with the result that sufficient accuracy may not be obtained in blood pressure measurement. In the case of 24-hour ambulatory continuous monitoring of the living body blood pressure values, when the cuff tightening force is increased until the blood flow stops at the artery of the living body limb, the living body may be subjected to heavy stress, which may offer insufficient accuracy in ambulatory blood pressure measurement values. Also there is a need for the cuff to compress the artery till the stop of the blood flow at the maximum blood pressure value of the living body and then to lower the compression pressure to the minimum blood pressure value, which takes time for a single intermittent measurement and makes the measurement noncontinuous, possibly rendering it impossible to detect blood pressure fluctuations in a shorter time span.

The present invention was conceived against the background of the above circumstances and an object thereof is to provide a blood pressure monitoring apparatus capable of alleviating the burden on the living body in the continuous blood pressure measurement, etc.

### Means for Solving Problem

The present inventors found out, during studying the relationship between the cuff compression pressure and pulse wave propagation velocity of the artery, that the relationship between the transmural pressure (intraarterial blood pressure-compression pressure) of the artery and the squared value of the pulse wave propagation velocity is expressed by a regression line within a range where the compression pressure is lower than the minimum blood pressure value of the living body. The present inventors further found out that the blood pressure value of the living body can be estimated by generating a proper relationship on the person to be measured among the maximum blood pressure value, the minimum blood pressure value, or the maximum blood pressure value and minimum blood pressure value, and the compression pressure and pulse wave propagation velocity related values, from the regression line, the real blood pressure value of the living body, and the real compression pressure and pulse wave propagation velocity, and applying plural sets of real compression pressures and pulse wave propagation velocities to the proper relationship. The present invention was conceived on the basis of such findings.

According to a first aspect of the invention, there is provided a blood pressure monitoring apparatus. The blood pressure monitoring apparatus has a cuff wrapped around a site to be compressed of a person to be measured to compress an artery of the person to be measured, the cuff having a plurality of inflatable bladders forming independent air chambers juxtaposed across width, the blood pressure monitoring apparatus repeatedly estimating an estimated arterial pressure of the person to be measured, and the blood pressure monitoring apparatus further comprises a linear relationship storage portion storing previously stored linear relationships between a plurality of transmural pressures of the artery that are pressure differences between an arterial pressure within the artery and a plurality of compression pressures of the cuff, and squared values of pulse wave propagation velocities respectively detected under the plurality of compression pressures of the cuff in a low pressure section lower than a diastolic arterial pressure of a living body, a blood pressure measurement portion measuring a real arterial pressure of the person to be measured, based on a pulse synchronous wave from the artery obtained in a pressure lowering process after compressing the site to be compressed of the person to be measured with a compression pressure higher than a systolic arterial pressure of the person to be measured, a proper relationship generation portion applying, for the person to be measured, the real arterial pressure, real compression pressures in the low pressure section, and real pulse wave propagation velocities based on propagation time between the pulse waves obtained respectively under the real compression pressures, to thereby generate a proper relationship on the person to be measured among the real arterial pressures of the person to be measured, the real compression pressures, and the real pulse wave propagation velocities, and a blood pressure estimation portion applying, for the person to be measured, the real compression pressures in the low pressure section and the real pulse wave propagation velocities obtained under the real compression pressures, to the proper relationship on the person to be measured, to thereby estimate the estimated arterial pressure.

According to a second aspect of the invention, in the blood pressure monitoring apparatus according to the first aspect of the invention,
the estimated arterial pressure estimated by the blood pressure estimation portion is an estimated diastolic arterial pressure DAPe of the person to be measured, and wherein
the linear relationship is a regression line expressed by Formula (1) below:

PWV²=s·(DAP−Pc)+i (1)
where PWV is the pulse wave propagation velocity of the living body, DAP is the diastolic arterial pressure of the living body, and Pc is the compression pressure on the living body, and
where s denotes a slope of the regression line and i denotes an intercept of the regression line.

According to a third aspect of the invention, in the blood pressure monitoring apparatus according to the second aspect of the invention,
the proper relationship on the person to be measured is expressed by Formula (2) below:

DAPe=PWV_{D}²/s_{D}−i_{D}/s_{D}+Pc (2)
where i_{D} and s_{D} are really measured calibration values, obtained respectively as solutions to unknowns i and s when: substituting, into two equations each expressed by Formula (1), a diastolic arterial pressure really measured on the person to be measured, as DAP; substituting thereinto different real compression pressures within the low pressure section, respectively, as Pc; and substituting thereinto real pulse wave propagation velocities based on propagation time between local minimum sites of pulse waves obtained respectively for the different real compression pressures, respectively, as PWV_{D}.

According to a fourth aspect of the invention, in the blood pressure monitoring apparatus according to the third aspect of the invention,
the propagation time between the local minimum sites of the pulse waves obtained respectively for the real compression pressures is propagation time between vertices occurring correspondingly to rising points of the pulse waves obtained respectively for the real compression pressures, in second derivative waveforms of the pulse waves obtained respectively for the real compression pressures.

According to a fifth aspect of the invention, in the blood pressure monitoring apparatus according to the third or fourth aspect of the invention,
the blood pressure estimation portion comprises a diastolic arterial pressure estimation portion estimating the estimated diastolic arterial pressure, by successively applying, for the person to be measured, real compression pressures in the low pressure section and the real pulse wave propagation velocities obtained under the real compression pressures, to the proper relationship of Formula (2).

According to a sixth aspect of the invention, in the blood pressure monitoring apparatus according to the first aspect of the invention,
the estimated arterial pressure estimated by the blood pressure estimation portion is an estimated systolic arterial pressure SAPe of the person to be measured, and wherein
the linear relationship is a regression line expressed by Formula (3) below:

PWV²=s·(SAP−Pc)+i (3)
where PWV is the pulse wave propagation velocity of the living body, SAP is the systolic arterial pressure of the living body, and Pc is the compression pressure on the living body, and
where s denotes a slope of the regression line and i denotes an intercept of the regression line.

According to a seventh aspect of the invention, in the blood pressure monitoring apparatus according to the sixth aspect of the invention,
the proper relationship on the person to be measured is expressed by Formula (4) below:

SAPe=PWV_{S}²/s_{S}−i_{S}/s_{S}+Pc (4)
where i_{S} and s_{S} are really measured calibration values, obtained as solutions to unknowns i and s when: substituting, into two equations each expressed by Formula (3), a systolic arterial pressure measured on the person to be measured, as SAP; substituting thereinto different real compression pressures within the low pressure section, respectively, as Pc; and substituting thereinto real pulse wave propagation velocities based on propagation time between local maximum sites of pulse waves obtained respectively for the different real compression pressures, respectively, as PWV_{S}.

According to an eighth aspect of the invention, in the blood pressure monitoring apparatus according to the seventh aspect of the invention,
the propagation time between local maximum sites of pulse waves obtained respectively for the real compression pressures is propagation time between local maximum points of pulse waves obtained respectively for the real compression pressures.

According to a ninth aspect of the invention, in the blood pressure monitoring apparatus according to the seventh or eighth aspect of the invention,
the blood pressure estimation portion comprises a systolic arterial pressure estimation portion estimating the estimated systolic arterial pressure, by successively applying, for the person to be measured, real compression pressures in the low pressure section and the real pulse wave propagation velocities obtained under the real compression pressures, to the proper relationship of Formula (4).

According to a tenth aspect of the invention, in the blood pressure monitoring apparatus according to the first aspect of the invention,
the estimated arterial pressure estimated by the blood pressure estimation portion is an estimated notch arterial pressure DNAPe of the person to be measured that is a compression pressure upon occurrence of notch sites locally formed posterior to local maximum sites of pulse waves obtained respectively for the real compression pressures, and wherein
the linear relationship is a regression line expressed by Formula (5) below.

PWV²=s·(DNAP−Pc)+i (5)
where PWV is the pulse wave propagation velocity of the living body, DNAP is the notch arterial pressure of the living body, and Pc is the compression pressure on the living body, and
where s denotes a slope of the regression line and i denotes an intercept of the regression line.

According to a eleventh aspect of the invention, in the blood pressure monitoring apparatus according to the tenth aspect of the invention,
the proper relationship on the person to be measured is expressed by Formula (6) below:

DNAPe=PWV_{DN}²/s_{DN}−i_{DN}/s_{DN}+Pc (6)
where i_{DN} and s_{DN} are really measured calibration values, obtained as solutions to unknowns i and s when: substituting, into two equations each expressed by Formula (5), a notch arterial pressure really measured on the person to be measured, as DNAP; substituting thereinto different real compression pressures within the low pressure section, respectively, as Pc; and substituting thereinto real pulse wave propagation velocities based on propagation time between notch sites of pulse waves obtained respectively for the different real compression pressures, respectively, as PWV_{DN}.

According to a twelfth aspect of the invention, in the blood pressure monitoring apparatus according to the eleventh aspect of the invention,
the propagation time between notch sites of the pulse waves obtained respectively for the real compression pressures is propagation time between vertices occurring posterior to time points corresponding to local maximum sites of pulse waves obtained respectively for the real compression pressures, in second derivative waveforms of the pulse waves obtained respectively for the real compression pressures.

According to a thirteenth aspect of the invention, in the blood pressure monitoring apparatus according to the eleventh or twelfth aspect of the invention,
the blood pressure estimation portion comprises a notch arterial pressure estimation portion estimating the estimated notch arterial pressure, by successively applying, for the person to be measured, real compression pressures in the low pressure section and the real pulse wave propagation velocities obtained under the real compression pressures, to the proper relationship of Formula (6).

According to a fourteenth aspect of the invention, in the blood pressure monitoring apparatus according to the thirteenth aspect of the invention, the blood pressure estimation portion comprises a diastolic arterial pressure estimation portion estimating an estimated diastolic arterial pressure of the person to be measured, by successively applying, for the person to be measured, real compression pressures in the low pressure section and real pulse wave propagation velocities obtained under the real compression pressures, to a proper relationship among the diastolic arterial pressures really measured on the person to be measured, the real compression pressures in the low pressure section, and the real pulse wave propagation velocities in the low pressure section; and a systolic arterial pressure estimation portion estimating an estimated systolic arterial pressure, by generating a relationship between magnitudes of pulse waves in the low pressure section and the estimated arterial pressures, based on the estimated diastolic arterial pressure estimated by the diastolic arterial pressure estimation portion and the estimated notch arterial pressure estimated by the notch arterial pressure estimation portion, and applying real maximum values of pulse waves successively obtained, to the relationship.

According to a fifteenth aspect of the invention, in the blood pressure monitoring apparatus according to any one of the first to fourteenth aspects of the invention, a compression pressure control portion stepwise lowers a plurality of compression pressures within the low pressure section so as to form a plurality of sections temporarily keeping the plurality of compression pressures at constant values in the low pressure section, a pulse wave extraction portion extracts pulse waves that are pressure oscillations occurring in synchronization with pulses within each of the plurality of inflatable bladders under compression pressures in the plurality of sections, and a pulse wave propagation velocity calculation portion calculates the pulse wave propagation velocity, based on time difference between pulse waves obtained in each of the plurality of sections and length between the plurality of inflatable bladders.

According to a sixteenth aspect of the invention, in the blood pressure monitoring apparatus according to any one of the first to fifteenth aspects of the invention, the cuff is wrapped around a site to be compressed of a living body and has an upstream inflatable bladder, an intermediate inflatable bladder, and a downstream inflatable bladder independent of each other and juxtaposed across width, each compressing the site to be compressed of the living body, and the artery within the site to be compressed is compressed with an equal compression pressure by the upstream inflatable bladder, the intermediate inflatable bladder, and the downstream inflatable bladder.

According to the first aspect of the invention, the blood pressure monitoring apparatus comprises the linear relationship storage portion storing previously stored linear relationships between a plurality of transmural pressures of the artery that are pressure differences between an arterial pressure within the artery and a plurality of compression pressures of the cuff, and squared values of pulse wave propagation velocities respectively detected under the plurality of compression pressures of the cuff in a low pressure section lower than a diastolic arterial pressure of a living body, the blood pressure measurement portion measuring a real arterial pressure of the person to be measured, based on a pulse synchronous wave from the artery obtained in a pressure lowering process after compressing the site to be compressed of the person to be measured with a compression pressure higher than a systolic arterial pressure of the person to be measured, the proper relationship generation portion applying, for the person to be measured, the real arterial pressure, real compression pressures in the low pressure section, and real pulse wave propagation velocities based on propagation time between the pulse waves obtained respectively under the real compression pressures, to thereby generate a proper relationship on the person to be measured among the real arterial pressures of the person to be measured, the real compression pressures, and the real pulse wave propagation velocities, and the blood pressure estimation portion applying, for the person to be measured, the real compression pressures in the low pressure section and the real pulse wave propagation velocities obtained under the real compression pressures, to the proper relationship on the person to be measured, to thereby estimate the estimated arterial pressure. In consequence, except when the blood pressure measurement portion measures the real arterial pressure of the person to be measured, the compression pressure of the cuff can be a lower value than the diastolic arterial pressure of the person to be measured, thus rendering it possible to alleviate the burden on the person to be measured and continuously measure the blood pressure.

According to the blood pressure monitoring apparatus of this embodiment, the proper relationship generation portion uses the real diastolic arterial pressure of the person to be measured, the real compression pressures and the pulse wave propagation velocities based on the propagation time between the local minimum sites of the pulse waves obtained under the real compression pressure, to generate a proper relationship of the person to be measured between the diastolic arterial pressure and the pulse wave propagation velocities, whereupon the arterial pressure estimation portion can easily estimate the estimated diastolic arterial pressure of the person to be measured by applying the real compression pressure obtained in the low pressure section lower than the diastolic arterial pressure and the pulse wave propagation velocity based on the time difference between the local minimum sites of the pulse waves obtained under the real compression pressure, to the proper relationship of the living body generated by the proper relationship generation portion.

According to the blood pressure monitoring apparatus of this embodiment, the propagation time is a propagation time between the respective rising points of the pulse waves. This makes it easy to obtain the propagation time between the local minimum sites of the pulse waves, leading to enhanced diastolic arterial pressure estimation of the person to be measured.

According to the fifth aspect of the invention, the blood pressure estimation portion includes the diastolic arterial pressure estimation portion that estimates the estimated diastolic arterial pressure of the person to be measured by successively applying, the real compression pressure in the low pressure section lower than the diastolic arterial pressure of the person to be measured and the real pulse wave propagation velocity obtained under the real compression pressure, to the proper relationship of Formula (2). This can alleviate the burden on the person to be measured, enabling easy estimation of the estimated diastolic arterial pressure of the person to be measured.

According to the sixth and seventh aspect of the invention, the proper relationship generation portion uses the real systolic arterial pressure of the person to be measured and the pulse wave propagation velocities based on the propagation time between the local maximum sites of the pulse waves obtained under the real compression pressures, to generate a proper relationship expression of the person to be measured between the systolic arterial pressure and the pulse wave propagation velocities, whereupon the systolic arterial pressure estimation portion can estimate the estimated systolic arterial pressure of the person to be measured by applying the real compression pressure obtained in the low pressure section lower than the diastolic arterial pressure and the pulse wave propagation velocity based on the time difference between the local maximum sites of the pulse waves obtained under the real compression pressure, to the proper relationship generated by the proper relationship generation portion.

According to the eighth aspect of the invention, the propagation time between the local maximum sites of the pulse waves obtained under the real compression pressure, respectively, is a propagation time between the local maximum points of the pulse waves obtained under the real compression pressure, respectively. This makes it easy to obtain the propagation time between the local maximum sites of the pulse waves, leading to enhanced blood pressure estimation accuracy.

According to ninth aspect of the invention, the blood pressure estimation portion includes the systolic arterial pressure estimation portion that estimates the estimated systolic arterial pressure of the person to be measured by successively applying the real compression pressure and the real pulse wave propagation time obtained under the real compression pressure, in the low pressure section, to the proper relationship of Formula (4), thus enabling easy estimation of the estimated systolic arterial pressure of the person to be measured.

According to the tenth and eleventh aspect of the invention, the proper relationship generation portion uses the real notch arterial pressure of the person to be measured and the pulse wave propagation velocities based on the propagation time between the notch sites of the pulse waves obtained under the real compression pressures, to generate a proper relationship expression of the person to be measured between the notch arterial pressure, the compression pressure, and the pulse wave propagation velocities. Whereupon the arterial pressure estimation portion can easily estimate the estimated notch arterial pressure of the person to be measured by applying the real compression pressure obtained in the low pressure section lower than the diastolic arterial pressure and the pulse wave propagation velocity based on the time difference between the notch sites of the pulse waves obtained under the real compression pressure, to the proper relationship generated by the proper relationship generation portion.

According to the twelfth aspect of the invention, the propagation time between the notch sites of the pulse waves obtained under the real compression pressure, respectively, is the propagation time between vertices occurring posterior to time points corresponding to local maximum sites of pulse waves obtained respectively for the real compression pressures, in second derivative waveforms of the pulse waves obtained respectively for the real compression pressures. This makes it easy to obtain the propagation time between the notch sites of the pulse waves, leading to enhanced notch arterial pressure estimation accuracy.

According to the thirteenth aspect of the invention, the arterial pressure estimation portion estimates the estimated notch arterial pressure of the person to be measured by successively applying the real compression pressure and the real pulse wave propagation velocity obtained under the real compression pressure, in the low pressure section, to the proper relationship of Formula (6), thus enabling easy estimation of the estimated notch arterial pressure of the person to be measured.

According to the fourteenth aspect of the invention, the blood pressure estimation portion comprises a diastolic arterial pressure estimation portion estimating an estimated diastolic arterial pressure of the person to be measured, by successively applying, for the person to be measured, real compression pressures in the low pressure section and real pulse wave propagation velocities obtained under the real compression pressures, to a proper relationship among the diastolic arterial pressures really measured on the person to be measured, the real compression pressures in the low pressure section, and the real pulse wave propagation velocities in the low pressure section; and a systolic arterial pressure estimation portion estimating an estimated systolic arterial pressure, by generating a relationship between magnitudes of pulse waves in the low pressure section and the estimated arterial pressures, based on the estimated diastolic arterial pressure estimated by the diastolic arterial pressure estimation portion and the estimated notch arterial pressure estimated by the notch arterial pressure estimation portion, and applying real maximum values of pulse waves successively obtained, to the relationship, thus enabling easy estimation of the estimated systolic arterial pressure of the person to be measured.

According to the fifteenth aspect of the invention, the blood pressure monitoring apparatus includes the compression pressure control portion that lowers stepwise the plurality of compression pressures in the low pressure section so as to form, in the low pressure section, the plurality of sections where the compression pressures are temporarily kept at constant values, the pulse wave extraction portion that extracts pulse waves as pressure oscillations generated in synchronization with pulses within the plurality of inflatable bladders, respectively, under the compression pressures in the plurality of sections, and the pulse wave propagation velocity calculation portion that calculates the pulse wave propagation velocity, based on the time difference between pulse waves obtained in each of the plurality of sections and the length between the plurality of inflatable bladders. Hence, the pulse waves obtained in each of the sections where the compression pressures are kept at constant values have waveforms without distortion caused by fluctuations of the compression pressure, ensuring correct obtainment of the pulse wave propagation velocity and correct calculation of the proper relationship.

According to the sixteenth aspect of the invention, the cuff is wrapped around the site to be compressed of the living body and has the independent upstream inflatable bladder, intermediate inflatable bladder, and downstream inflatable bladder juxtaposed across the width and each compressing the site to be compressed of the living body, the upstream inflatable bladder, intermediate inflatable bladder, and downstream inflatable bladder each compresses the artery within the site to be compressed at the same compression pressure. This is advantageous in that the blood pressure measurement using compression on the four limbs of the living body and the detection of the pulse wave propagation velocity can be performed at the same time.

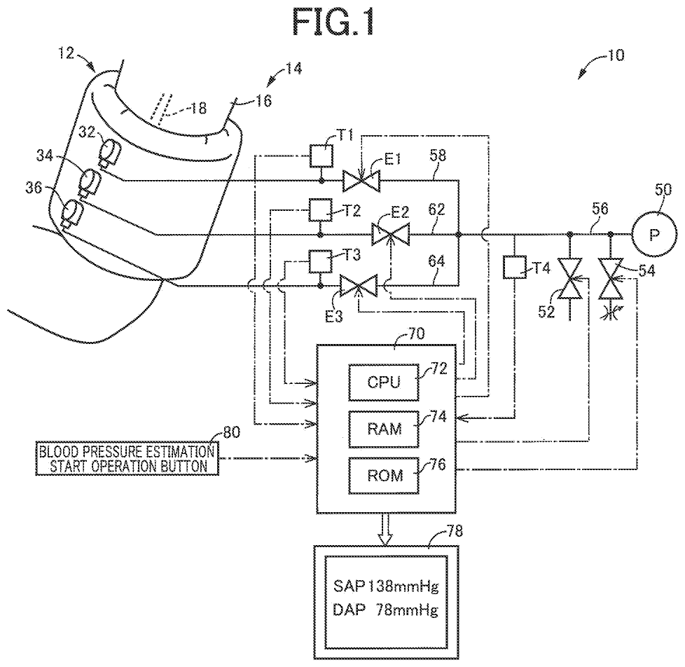

FIG. 1 shows a block diagram of the blood pressure monitoring apparatus according to an embodiment of the invention.

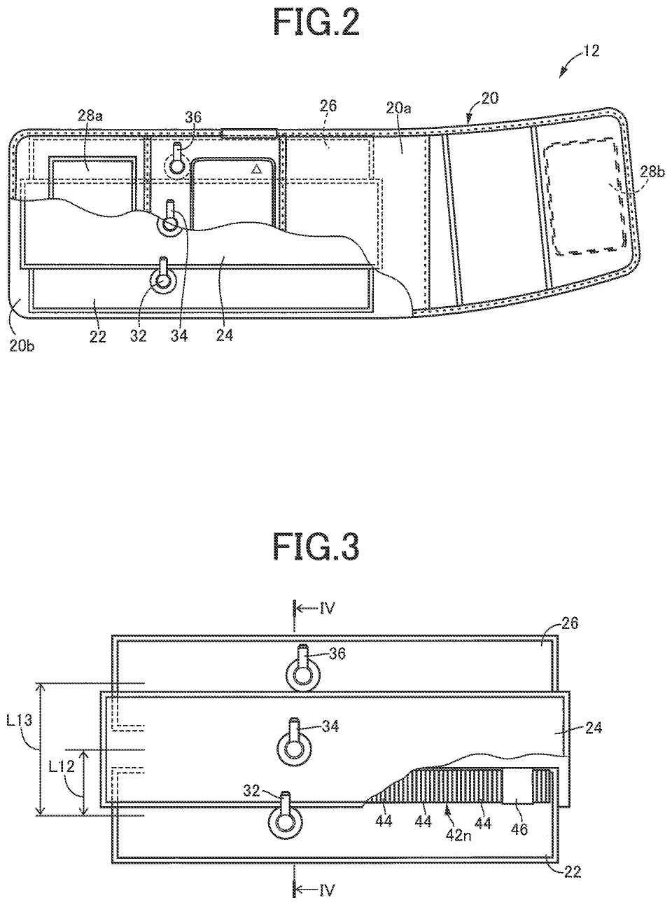

FIG. 2 is a view showing the cuff with an outer-peripheral-side partly cut out.

FIG. 3 is a plan view showing the upstream inflatable bladder, the intermediate inflatable bladder, and the downstream inflatable bladder, disposed within the cuff in the FIG. 2.

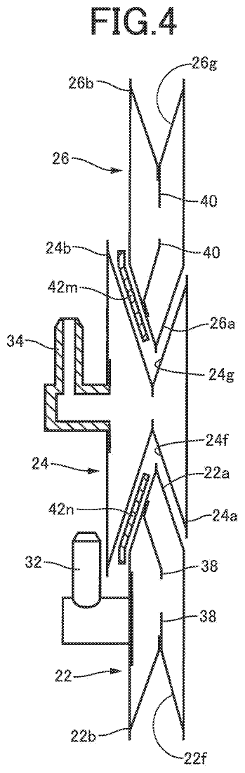

FIG. 4 is a sectional view taken along line IV-IV of FIG. 3, and the upstream inflatable bladder, the intermediate inflatable bladder, and the downstream inflatable bladder are shown cut out in the width direction.

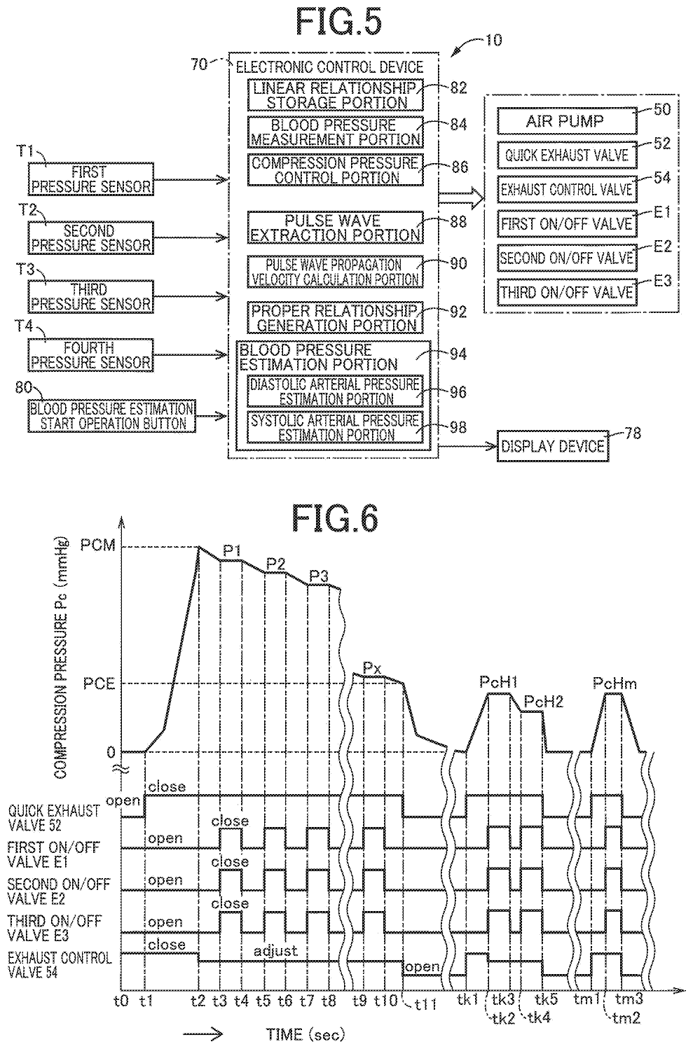

FIG. 5 is a function block diagram for explaining a principal part of control function provided by the electronic control device in FIG. 1.

FIG. 6 is a timechart explaining a principal part of compression pressure control action of the cuff by the compression pressure control portion in FIG. 5.

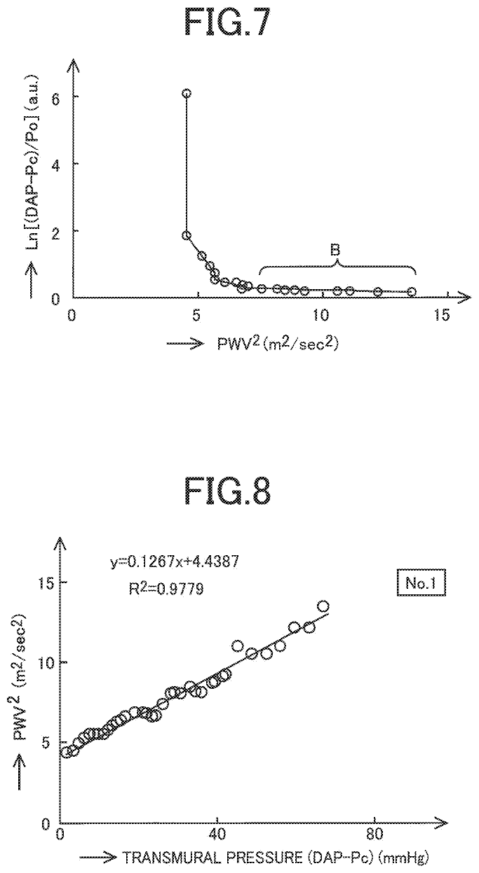

FIG. 7 shows two-dimensional coordinates explaining relationship between the squared value of the pulse propagation velocity PWV² and Ln((DAP−Pc)/Po), when changing the compression pressure Pc in the all range where the compression pressure Pc is less than the diastolic arterial pressure DAP.

FIG. 8 is a diagram showing the experimental results for one experimental animal (dog), conducted by the present inventors, showing, on the two-dimensional coordinates, the relationship between DAP−Pc and the squared value of the pulse propagation velocity PWV², when changing the compression pressure Pc in the all range where the compression pressure Pc is less than the diastolic arterial pressure DAP.

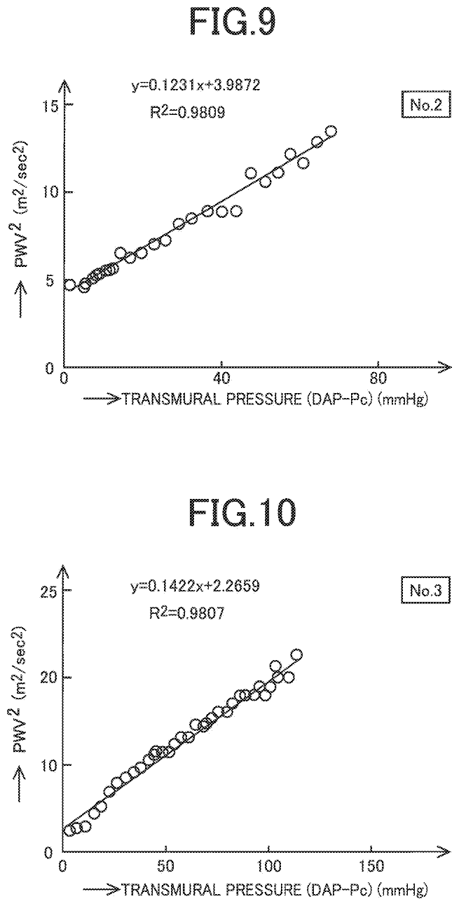

FIG. 9 is a diagram showing the result of Experiment No. 2 similarly as FIG. 8, conducted by the present inventors, for the same living body as in FIG. 8, when raised the arterial pressure.

FIG. 10 is a diagram showing the result of Experiment No. 3 similarly as FIG. 8, conducted by the present inventors, for the same living body as in FIGS. 8 and 9, when raised the arterial pressure.

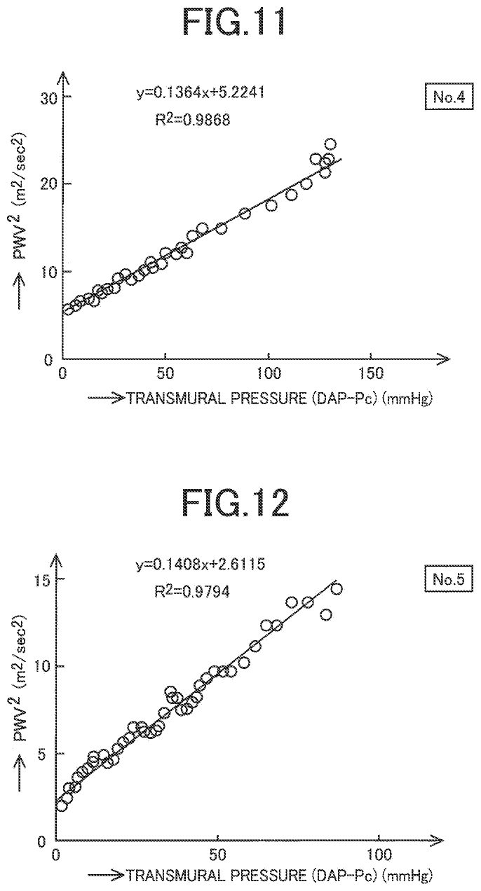

FIG. 11 is a diagram showing the result of Experiment No. 4 similarly as FIG. 8, conducted by the present inventors, for the same living body as in FIGS. 8 to 10, when raised the arterial pressure.

FIG. 12 is a diagram showing the result of Experiment No. 5 similarly as FIG. 8, conducted by the present inventors, for the same living body as in FIGS. 8 to 11, when returned the arterial pressure to the original state.

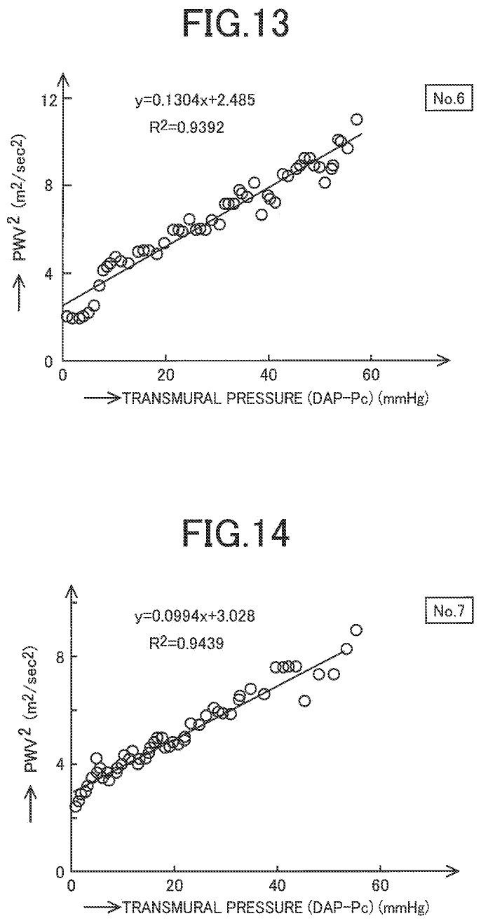

FIG. 13 is a diagram showing the result of Experiment No. 6 similarly as FIG. 8, conducted by the present inventors, for the same living body as in FIGS. 8 to 12, when lowered the arterial pressure.

FIG. 14 is a diagram showing the result of Experiment No. 7 similarly as FIG. 8, conducted by the present inventors, for the same living body as in FIGS. 8 to 13, when lowered the arterial pressure.

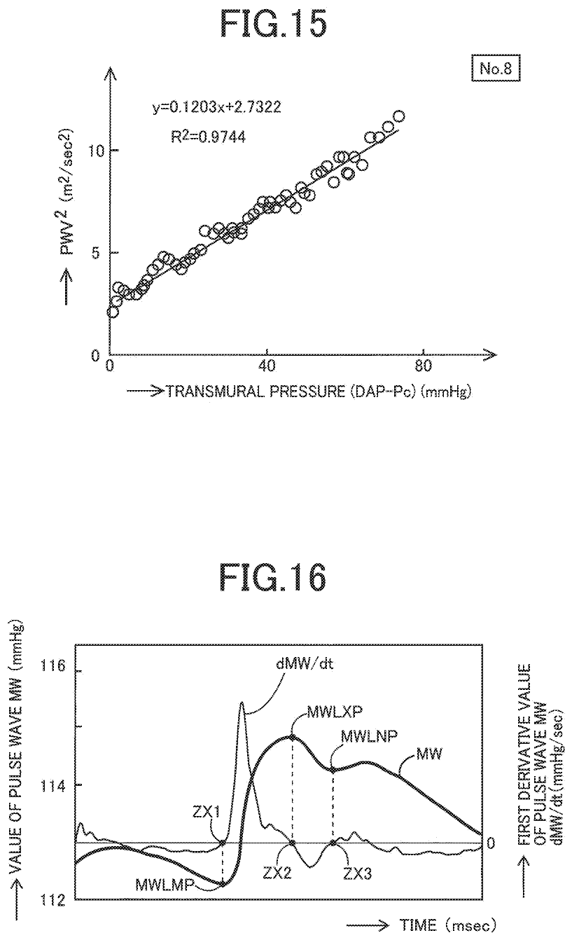

FIG. 15 is a diagram showing the result of Experiment No. 8 similarly as FIG. 8, conducted by the present inventors, for the same living body as in FIGS. 8 to 14, when returned the arterial pressure to the original state.

FIG. 16 is a diagram indicating the pulse wave and its first derivative waveform that are superimposed in phase on a common time axis, and shows the correspondence between a local minimum site MWLMP of the pulse wave, a local maximum site MWLXP of the pulse wave, and a notch site MWLNP of the pulse wave; and a zero-crossing points ZX1, ZX2, and ZX3 of the first derivative waveform of the pulse wave.

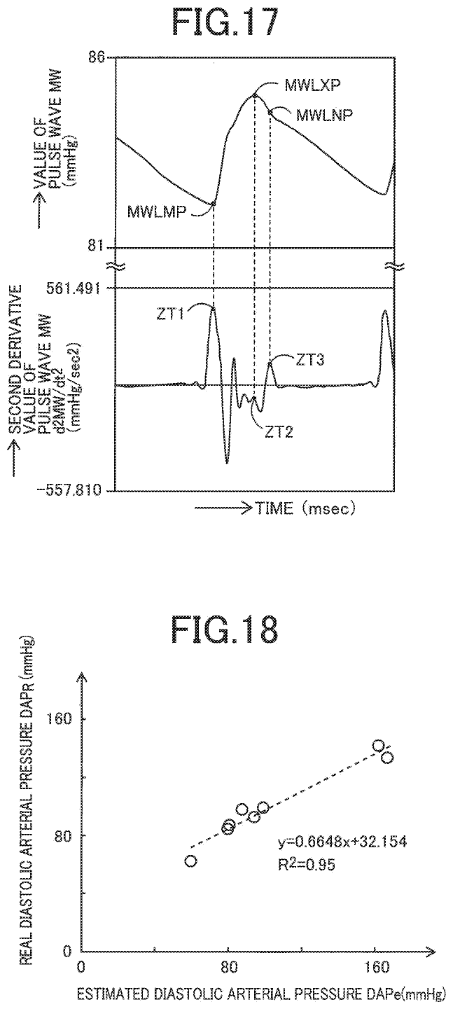

FIG. 17 is a diagram indicating the pulse wave and the second derivative waveform of the pulse wave in phase on a common time axis, and shows the correspondence between a local minimum site MWLMP of the pulse wave, a notch site MWLNP of the pulse wave, and a local maximum site MWLXP of the pulse wave; and the vertex ZT1, the vertex ZT3 of the second derivative waveform of the pulse wave, and the vertex ZT2 lying at the same time point as that of MWLXP.

FIG. 18 is a diagram indicating the experimental results conducted by the present inventors showing relationship between: the estimated diastolic arterial pressures and the really measured diastolic arterial pressures by the control action of electronic control device in FIG. 1.

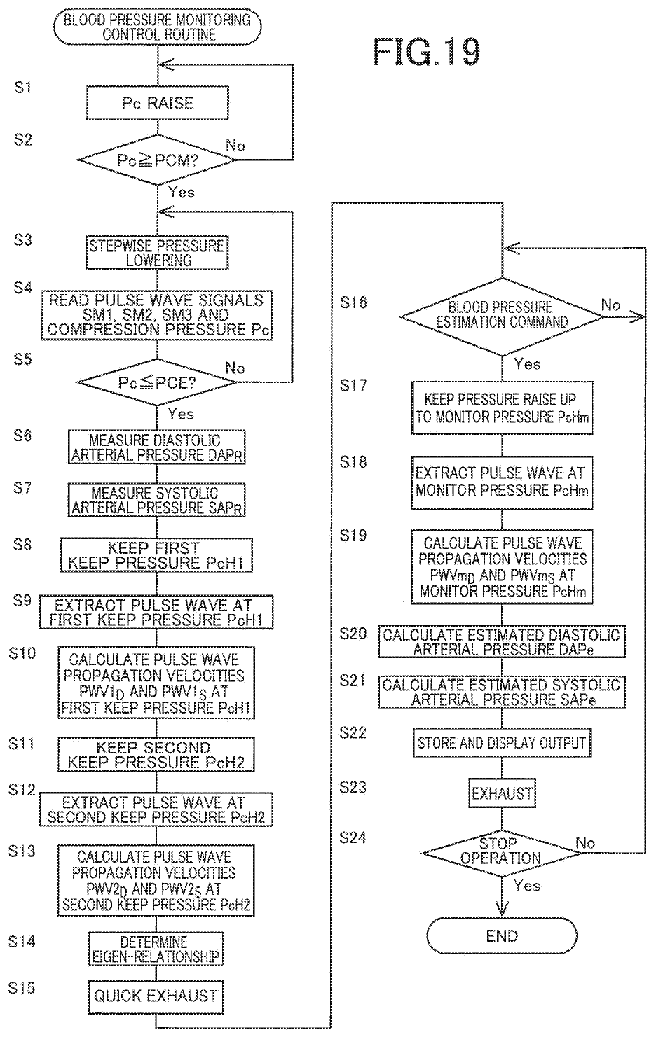

FIG. 19 is a flowchart explaining a control action of the electronic control device.

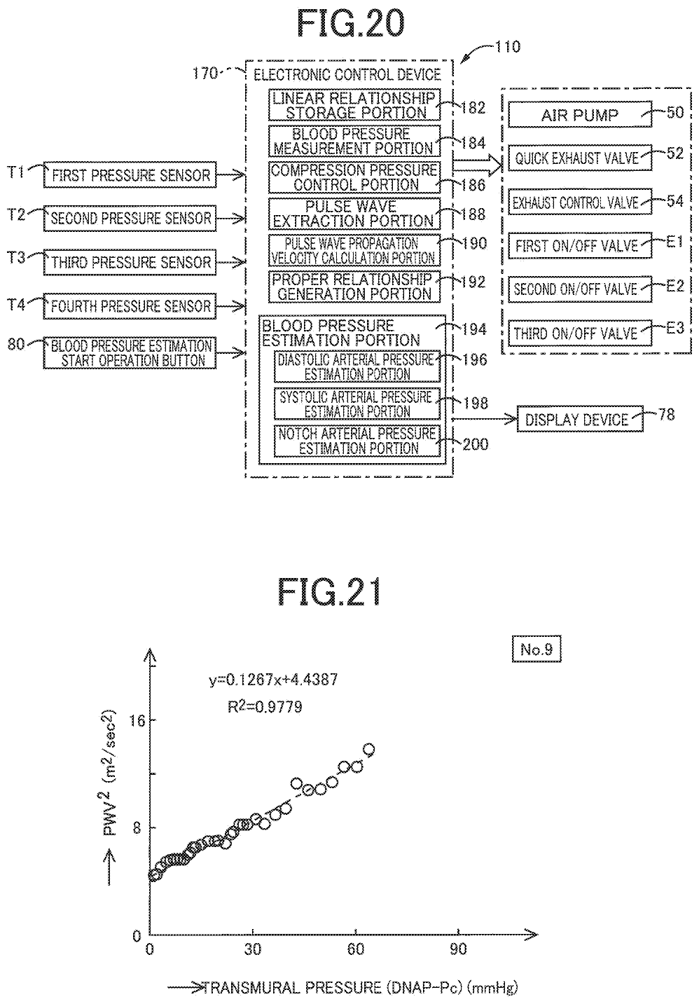

FIG. 20 is a function block diagram for explaining a principal part of control function provided by the electronic control device in other embodiment of the invention, which corresponds to FIG. 5.

FIG. 21 is a diagram showing, together with the regression line y and the determination coefficient R², two-dimensional coordinate data indicative of results of Experiment No. 9 performed by the present inventors.

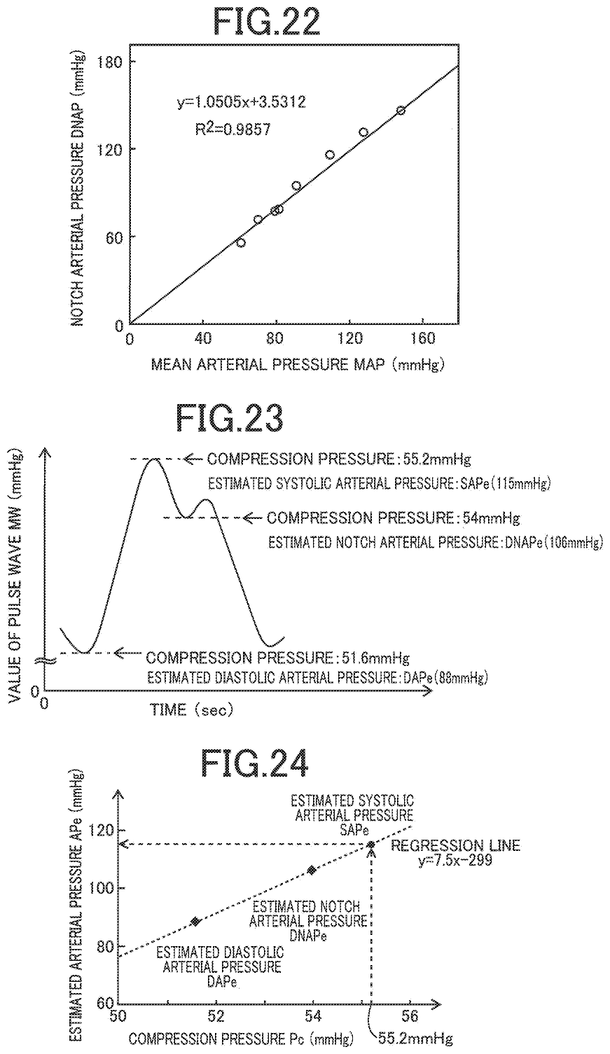

FIG. 22 shows the correlation on an animal (dog) between the notch arterial pressure DNAP directly measured using a catheter and the mean arterial pressure really measured.

FIG. 23 is a diagram explaining the relationship between the local minimum site, notch site, local maximum site, and the estimated diastolic arterial pressure, estimated notch arterial pressure, estimated systolic arterial pressure of the pulse wave obtained in the monitor pressure keep section.

FIG. 24 shows the relationship obtained in advance, for the living body to be measured so as to estimate the estimated systolic arterial pressure in the embodiments in FIG. 20.

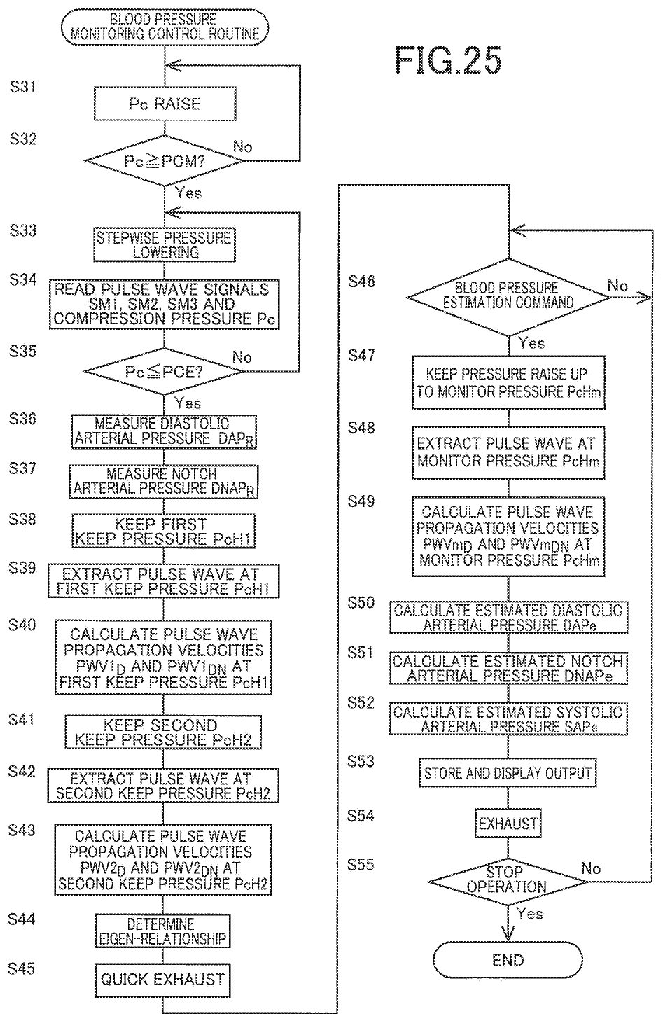

FIG. 25 is a flowchart explaining a principal part of the control action of the electronic control device according to the embodiments in FIG. 20.

### MODES FOR CARRYING OUT THE INVENTION

Embodiments of the present invention will hereinafter be described in detail with reference to the drawings. In the following embodiments, the figures are appropriately simplified or modified, and the dimension ratios, shapes, etc. of parts are not necessarily correctly drawn.

### First Embodiment

FIG. 1 shows a blood pressure monitoring apparatus 10 (automatic blood pressure measuring apparatus) working also as a blood pressure estimator of an example of the present invention that includes a cuff 12 for upper arm wrapped around a compressed area e.g. an upper arm 16 that is a living body limb such as an arm or an ankle of a living body 14 as a person to be measured. In the process of lowering a compression pressure Pc of the cuff 12 raised to a value enough to stop flowing of an artery 18 within the upper arm 16, this blood pressure monitoring apparatus 10 sequentially extracts pulse waves that are pressure oscillations of the compression pressure Pc within the cuff 12 generated in response to volume changes in the artery 18, and measures a systolic arterial pressure SAP and a diastolic arterial pressure DAP of the living body 14, based on information obtained from the pulse waves.

FIG. 2 is a view showing the cuff 12 with an outer-peripheral-side nonwoven fabric 20a partly cut out. As shown in FIG. 2, the cuff 12 includes: a belt-shaped outer bladder 20 composed of the outer-peripheral-side nonwoven fabric 20a and an inner-peripheral-side nonwoven fabric 20b, made of synthetic resin fibers and having back sides mutually laminated with synthetic resin such as PVC (polyvinyl chloride); and an upstream inflatable bladder 22, an intermediate inflatable bladder 24, and a downstream inflatable bladder 26, which are made from e.g. a flexible sheet such as a soft polyvinyl chloride and housed in sequence in the width direction within the belt-shaped outer bladder 20, the bladders 22, 24, and 26 capable of independently compressing the upper arm 16. This cuff 12 is detachably attached to the upper arm 16 by removably fastening a brushed pile 28b secured to an end of the inner-peripheral-side nonwoven fabric 20b onto a hook-and-loop fastener 28a secured to an end of the outer-peripheral-side nonwoven fabric 20a.

The upstream inflatable bladder 22, the intermediate inflatable bladder 24, and the downstream inflatable bladder 26 are juxtaposed across the width of the elongated cuff 12 and each have an independent air chamber separately compressing the upper arm 16, the bladders 22, 24, and 26 comprising tube connecting connectors 32, 34, and 36, respectively, on the outer peripheral side. Those tube connecting connectors 32, 34, and 36 are exposed on the outer peripheral surface of the cuff 12 through the outer-peripheral-side nonwoven fabric 20a.

FIG. 3 is a plan view showing the upstream inflatable bladder 22, the intermediate inflatable bladder 24, and the downstream inflatable bladder 26, disposed within the cuff 12, and FIG. 4 is a sectional view taken along line V-IV of FIG. 3. The upstream inflatable bladder 22, the intermediate inflatable bladder 24, and the downstream inflatable bladder 26 each have an elongated shape and serve to detect pulse waves that are pressure oscillations generated in response to volume changes in the artery 18 compressed thereby. The upstream inflatable bladder 22 and the downstream inflatable bladder 26 are arranged adjacent to both sides of the intermediate inflatable bladder 24, and the intermediate inflatable bladder 24 is arranged at the central portion in the width direction of the cuff 12, sandwiched between the upstream inflatable bladder 22 and the downstream inflatable bladder 26. The center of the upstream inflatable bladder 22 and the center of the intermediate inflatable bladder 24 are apart a distance L12 from each other, while the center of the upstream inflatable bladder 22 and the center of the downstream inflatable bladder 26 are apart a length L13 from each other. With the cuff 12 being wrapped around the upper arm 16, the upstream inflatable bladder 22 and the downstream inflatable bladder 26 are positioned with a predetermined interval in the longitudinal direction of the upper arm 16, while the intermediate inflatable bladder 24 is arranged between the upstream inflatable bladder 22 and the downstream inflatable bladder 26 such that it is juxtaposed in the longitudinal direction of the upper arm 16.

The intermediate inflatable bladder 24 has, at its both sides, a side edge portion of a so-called gusset structure. That is, at both ends in the longitudinal direction of the upper arm 16 i.e. in the width direction of the cuff 12, the intermediate inflatable bladder 24 has a pair of folding grooves 24f and 24g, respectively, formed from a flexible sheet that are folded in the direction of approaching each other so that the closer they are to each other, the deeper they are. The upstream inflatable bladder 22 and the downstream inflatable bladder 26 are arranged such that their respective ends 22a and 26a on the side adjacent to the intermediate inflatable bladder 24 are received in the pair of folding grooves 24f and 24g, respectively. This provides an overlapping structure i.e. a structure in which the end 24a of the intermediate inflatable bladder 24 and the end 22a of the upstream inflatable bladder 22 overlap each other, with the end 24b of the intermediate inflatable bladder 24 and the end 26a of the downstream inflatable bladder 26 overlapping each other, whereupon, when the upstream inflatable bladder 22, the intermediate inflatable bladder 24, and the downstream inflatable bladder 26 compress the upper arm 16 with equal pressure, uniform pressure distribution can be obtained even in the vicinity of their boundaries.

The upstream inflatable bladder 22 and the downstream inflatable bladder 26 also have a side edge portion of a gusset structure at their respective ends 22b and 26b opposite to the intermediate inflatable bladder 24. That is, the upstream inflatable bladder 22 has, at its end 22b opposite to the intermediate inflatable bladder 24, a folding groove 22f, formed from a flexible sheet that is folded in the direction of approaching each other so that the closer they are to each other, the deeper it is. The downstream inflatable bladder 26 has, at its end 26b opposite to the intermediate inflatable bladder 24, a folding groove 26g, formed from a flexible sheet that is folded in the direction of approaching each other so that the closer they are to each other, the deeper it is. To prevent protruding in the width direction of the cuff 12, the sheet forming the folding groove 22f is connected, via a connecting sheet 38 with a through hole arranged within the upstream inflatable bladder 22, to its opposite portion i.e. its portion toward the intermediate inflatable bladder 24. Similarly, the sheet forming the folding groove 26g is connected, via a connecting sheet 40 with a through hole arranged within the downstream inflatable bladder 26, to its opposite portion i.e. its portion toward the intermediate inflatable bladder 24.

This allows the compression pressure Pc on the artery 18 of the upper arm 16 to be equally applied to ends 22b and 26b of the upstream inflatable bladder 22 and the downstream inflatable bladder 26 as well as other portions, so that the effective compression width in the width direction of the cuff 12 equals its width dimension. Due to the structure of the cuff 12 in which the three bladders i.e. the upstream inflatable bladder 22, the intermediate inflatable bladder 24, and downstream inflatable bladder 26 are arranged in the width direction dimension of about 12 cm, each bladder has to be substantially about 4 cm wide. To sufficiently generate the compression function even with such a narrow width dimension, the overlapping structure is provided in which the both ends 24a and 24b of the intermediate inflatable bladder 24 and the ends 22a and 26a of the upstream inflatable bladder 22 and the downstream inflatable bladder 26 overlap each other respectively, with the ends 22b and 26b opposite to the intermediate inflatable bladder 24 of the upstream inflatable bladder 22 and the downstream inflatable bladder 26 being formed as the side edge portions of the so-called gusset structure.

Elongated shielding members 42n and 42m are respectively interposed between the ends 22a and 26a toward the intermediate inflatable bladder 24 of the upstream inflatable bladder 22 and the downstream inflatable bladder 26 and inner wall surfaces i.e. opposed groove side surfaces of the pair of folding grooves 24f and 24g receiving the ends 22a and 26a, the shielding members 42n and 42m having stiffness anisotropy that the bending stiffness in the width direction of the cuff 12 is higher than the bending stiffness in the longitudinal direction of the cuff 12. The shielding member 42n has a length dimension similar to the overlap dimension between the upstream inflatable bladder 22 and the intermediate inflatable bladder 24. Similarly, the shielding member 42m has a length dimension similar to the overlap dimension between the downstream inflatable bladder 26 and the intermediate inflatable bladder 24.

As shown in FIGS. 3 and 4, the elongated shielding members 42n and 42m are interposed respectively in a gap on the outer peripheral side of gaps between the end 22a of the upstream inflatable bladder 22 and the folding groove 24f receiving the end 22a and in a gap on the outer peripheral side of gaps between the end 26a of the downstream inflatable bladder 26 and the folding groove 24g receiving the end 26a. Although in this embodiment, the elongated shielding members 42n and 42m are disposed in the gaps on the outer peripheral side since the shielding effect is greater in the gaps on the outer peripheral side than in the gaps on the inner peripheral side, they may be disposed both in the gaps on the outer peripheral side and in the gaps on the inner peripheral side.

The shielding members 42n and 42m include a plurality of flexible hollow tubes 44 made of resin parallel to each other in the longitudinal direction of the upper arm 16 (i.e. in the width direction of the cuff 12), the flexible hollow tubes 44 being arranged, in parallel to each other, juxtaposed in the circumferential direction of the upper arm 16 (i.e. in the longitudinal direction of the cuff 12), the flexible hollow tubes 44 being coupled to each other directly by molding or adhesion or indirectly via another member such as an adhesive tape or other flexible sheet, to form the shielding members 42n and 42m. The shielding member 42n is hooked on a plurality of hooking sheets 46 disposed at a plurality of locations on the outer peripheral side of the end 22a toward the intermediate inflatable bladder 24 of the upstream inflatable bladder 22. In the same manner, the shielding member 42m is hooked on the plurality of hooking sheets 46 disposed at a plurality of locations on the outer peripheral side of the end 26a toward the intermediate inflatable bladder 24 of the downstream inflatable bladder 26.

Referring back to FIG. 1, in the blood pressure monitoring apparatus 10, an air pump 50, a quick exhaust valve 52, and an exhaust control valve 54 are each connected to a main tube 56. From the main tube 56 are each branched off a first branch tube 58 connected to the upstream inflatable bladder 22, a second branch tube 62 connected to the intermediate inflatable bladder 24, and a third branch tube 64 connected to the downstream inflatable bladder 26. The first branch tube 58 includes a first on/off valve E1 for direct on/off between the air pump 50 and the upstream inflatable bladder 22. The second branch tube 62 includes a second on/off valve E2 for direct on/off between the air pump 50 and the intermediate inflatable bladder 24. The third branch tube 64 includes a third on/off valve E3 for direct on/off between the air pump 50 and the downstream inflatable bladder 26.

A first pressure sensor T1 for detecting a pressure value within the upstream inflatable bladder 22 is connected to the first branch tube 58: a second pressure sensor T2 for detecting a pressure value within the intermediate inflatable bladder 24 is connected to the second branch tube 62; a third pressure sensor T3 for detecting a pressure value within the downstream inflatable bladder 26 is connected to the third branch tube 64; and a fourth pressure sensor T4 for detecting a compression pressure Pc of the cuff 12 is connected to the main tube 56.

An electronic control device 70 is fed with: an output signal indicative of a pressure value within the upstream inflatable bladder 22 i.e. a compression pressure Pc1 of the upstream inflatable bladder 22 from the first pressure sensor T1; an output signal indicative of a pressure value within the intermediate inflatable bladder 24 i.e. a compression pressure Pc2 of the intermediate inflatable bladder 24 from the second pressure sensor T2; an output signal indicative of a pressure value within the downstream inflatable bladder 26 i.e. a compression pressure Pc3 of the downstream inflatable bladder 26 from the third pressure sensor T3; and an output signal indicative of a compression pressure Pc of the cuff 12 from the fourth pressure sensor T4.

The electronic control device 70 is a so-called microcomputer that includes a CPU 72, a RAM 74, a ROM 76, a display device 78, an I/O port not shown, etc. In this electronic control device 70, the CPU 72 processes input signals in accordance with a program previously stored in the ROM 76 while utilizing the storage function of the RAM 74, and controls each of the electromotive air pump 50, the quick exhaust valve 52, the exhaust control valve 54, the first on/off valve E1, the second on/off valve E2, and the third on/off valve E3, in response to the operation of a blood pressure estimation start operation button 80, thereby executing automatic blood pressure measurement control to allow display of the measurement results on the display device 78.

FIG. 5 is a function block diagram for explaining a principal part of control function provided by the electronic control device 70. In FIG. 5, the electronic control device 70 functionally includes a linear relationship storage portion 82, a blood pressure measurement portion 84, a compression pressure control portion 86, a pulse wave extraction portion 88, a pulse wave propagation velocity calculation portion 90, a proper relationship generation portion 92, and a blood pressure estimation portion 94 having a diastolic arterial pressure estimation portion 96 and a systolic arterial pressure estimation portion 98. FIG. 6 is a timechart explaining a principal part of compression pressure control action of the cuff 12 by the compression pressure control portion 86.

The linear relationship storage portion 82 stores in advance stored linear relationships between squared values PWV² of a plurality of pulse wave propagation velocities PWV respectively detected under a plurality of compression pressures Pc of the cuff 12 in a low pressure section lower than the diastolic arterial pressure DAP of the living body 14, and transmural pressures (AP−Pc) of the artery 18 that are pressure differences between arterial pressures AP within the artery 18 and the compression pressures Pc. Specifically, for the diastolic arterial pressure DAP, a regression line representing a linear relationship expressed by Formula (1) is stored, whereas for the systolic arterial pressure SAP, a regression line representing a linear relationship expressed by Formula (3) is stored.

PWV²=s·(DAP−Pc)+i (1)

PWV²=s·(SAP−Pc)+i (3)
where s denotes a slope of the regression line and i denotes an intercept of the regression line.

In the following, the regression line will be described. Bramwell Hill's formula expressed in Formula (7) is generally known for the pulse wave propagation velocity within the artery. In Formula (7), V denotes an arterial volume, P denotes an intra-arterial blood pressure, and ρ denotes a blood density. Here let A be a blood vessel cross-sectional area and let L be a length between inflated bladders, then the arterial volume V is expressed by Formula (8), and differentiating both sides of Formula (8) with respect to A gives Formula (9).

PWV=√((V·dP)/(ρ·dV)) (7)

V=A·L (8)

dV=dA·L (9)

For the blood pressure P and the blood vessel cross-sectional area A, an exponential function model formula is established that includes an exponential function constant Po and coefficient α expressed in Formula (10), which Formula (10) is rewritten as Formula (11). Here for simplicity, assuming the density ρ to be 1, the relationship between the pulse wave propagation velocity PWV and the blood pressure P is expressed by Formula (12), based on Formulae (7), (9), and (11).

P=Po·e^{αA} (10)

dP=α·P·dA (11)

PWV²=P·Ln(P/Po) (12)

Assuming the diastolic arterial pressure DAP of a living body to be stable, when changing the compression pressures Pc of the cuff 12 within the pressure range (low pressure section) lower than the diastolic arterial pressure DAP of the living body, the transmural pressure (DAP−Pc) that is a pressure difference applied to the blood vessel wall of the artery 18 and the pulse wave propagation velocity PWV change while responding sequentially in individual pulses. Hence, in a certain pulse, Formula (12) is rewritten as expression model Formula (13) which follows.

PWV²=(DAP−Pc)·Ln((DAP−Pc)/Po) (13)
where Pc<DAP

The present inventors found out that the relationship in Formula (13) between Ln((DAP−Pc)/Po that is a term including Po in the right-hand side and the left-hand side PWV² is unvarying when the compression pressure Pc lies within a range of 20 mm Hg to 60 mm Hg, i.e., a range B shown in FIG. 7 with the diastolic arterial pressure DAP stabilized. FIG. 7 shows two-dimensional coordinates having a horizontal axis representative of the squared value PWV² and a vertical axis representative of Ln((DAP−Pc)/Po), and shows a curved line obtained by calculating PWV² and Ln((DAP−Pc)/Po) from measurement data of the pulse wave propagation velocity PWV when changing the compression pressure Pc in all the range where the compression pressure Pc is equal to or less than the diastolic arterial pressure DAP. Then, in a range B of pressure sufficiently lower than the diastolic arterial pressure DAP, e.g., a range of 20 mm Hg to 60 mm Hg, Ln((DAP−Pc)/Po) is substantially constant.

In the three-stranded cuff 12 having the independent upstream inflatable bladder 22, intermediate inflatable bladder 24, and downstream inflatable bladder 26 juxtaposed in the width direction, simultaneous measurement is possible of a phase difference (propagation time) Δt at a rising point of the pulse wave obtained from each of the pair of upstream inflatable bladder 22 and downstream inflatable bladder 26, and the compression pressure Pc at that time, in a stepwise pressure lowering process where a constant pressure is kept at plural different stages within a pressure range in which the compression pressure Pc is lower than the diastolic arterial pressure DAP of the living body 14. Since the length L13 between the pair of upstream inflatable bladder 22 and downstream inflatable bladder 26 is known, the pulse wave propagation velocity PWV(=L13/Δt) can be figured out sequentially. Then, for the term Ln((DAP−Pc)/Po) in Formula (13), Formula (13) is rewritten with a constant value x as expressed by Formula (14) in the pressure range (low pressure section) lower than the diastolic arterial pressure DAP of the living body, e.g., in a low range where the compression pressure Pc is 20 to 60 mm Hg.

PWV²∝κ·(DAP−Pc) (14)

Further generalization of the relationship in Formula (14) between the pulse wave propagation velocity PWV and the transmural pressure (DAP−Pc) gives Formula (1), i.e., the regression line with the slope s and the intercept i. By really measuring a diastolic arterial pressure DAP_{R} in advance for a predetermined person to be measured and then by substituting, into two equations same as Formula (1), plural pairs of compression pressure Pc and pulse wave propagation velocity PWV each pair measured at mutually different plural compression pressures Pc within the pressure range (low pressure section) lower than the diastolic arterial pressure DAP_{R} of the person to be measured, i_{D} and s_{D} are obtained as solutions to two unknowns i and s, respectively, of those simultaneous equations, and using i_{D} and s_{D} as really measured calibration values, a proper relationship expressed by Formula (2) described later is obtained.

The present inventors conducted experiments to obtain a regression line between the transmural pressure (DAP−Pc) and the squared value PWV² of the pulse wave propagation velocity PWV, by measuring the diastolic arterial pressure DAP_{R} of the same living body (dog), using an intravascular catheter for blood pressure measurement, at eight time points when blood pressure was extensively altered by drugs and then by calculating the transmural pressure (DAP−Pc) from plural sets of data including mutually different plural compression pressures Pc within the low pressure section lower than those diastolic arterial pressures and a plurality of pulse wave propagation velocities measured under the compression pressures.

FIGS. 8 to 15 are diagrams showing, on the two-dimensional coordinates, the relationship between the transmural pressure (DAP−Pc) and the squared value PWV², based on plural pieces of data obtained, using the intravascular catheter for blood pressure measurement, at eight time points (eight experiments No. 1 to N. 8) where blood pressure was extensively altered by drugs in one experimental animal (dog), conducted by the present inventors. As seen in FIGS. 8 to 15, in any one of the experiments No. 1 to No. 8, the value of a determination coefficient R² of a regression line y was 0.94 to 0.99 approximating 1, and a high-quality linear relationship was obtained. That is, it was confirmed that the regression line represented by Formula (1) can be stably obtained even if the blood pressure is greatly fluctuated.

Prior to generation of the proper relationship of Formula (2) by the proper relationship generation portion 92, the blood pressure measurement portion 84 measures an real systolic arterial pressure SAP_{R} and an real diastolic arterial pressure DAP_{R} of the person to be measured. In this blood pressure measurement, the compression pressure Pc of the cuff 12 is raised up to a pressure-raise target value higher than the systolic arterial pressure of the person to be measured, by the compression pressure control portion 86, in accordance with e.g. the well-known oscillometric method, after which in the pressure lowering process where the compression pressure Pc is gradually lowered, a pulse wave is detected that is superimposed on the compression pressure Pc2 of the intermediate inflatable bladder 24 and that pulsates in synchronization with the pulse, whereupon the systolic arterial pressure SAP_{R} and the DAP_{R} are determined based on the compression pressure Pc corresponding to an inflection point of an envelope joining the maxima of the amplitude of the pulse wave. In this blood pressure measurement, the real systolic arterial pressure SAP_{R} and the real diastolic arterial pressure DAP_{R} may be determined based on the compression pressure Pc when there occurs a vascular sound (Korotkoff sound) and the compression pressure Pc when there disappears the vascular sound that is generated in synchronization with a pulse detected by a microphone in the pressure lowering process, in accordance with e.g. the well-known Korotkoff sound method. The pulse wave and the vascular sound is a pulse synchronous wave generated in synchronization with the pulse of a living body.

In response to the operation of the blood pressure estimation start operation button 80, the compression pressure control portion 86 first closes the quick exhaust valve 52 and the exhaust control valve 54 and opens the first on/off valve E1, the second on/off valve E2, and the third on/off valve E3 to activate the air pump 50, for measurement by the blood pressure measurement portion 84 to obtain the real arterial pressure AP_{R} of the living body 14 as the person to be measured, whereby the compression pressure Pc on the living body 14 of the cuff 12 is quickly raised up to a pressure sufficiently higher than the systolic arterial pressure SAP of the living body 14, e.g., a pressure-raise target pressure value PCM preset to 180 mm Hg.

The compression pressure control portion 86 then repeatedly opens the exhaust control valve 54 at a predetermined cycle during a predetermined period of time to thereby gradually lower the compression pressure Pc of the cuff 12 in a stepwise manner at a previously set pressure lowering velocity until the compression pressure Pc of the cuff 12 becomes smaller than a measurement end pressure value PCE such that a plurality of constant step pressures P1, P2, P3, . . . , Px are kept in sequence for the duration until the compression pressure Pc of the cuff 12 reaches a pressure sufficiently lower than the diastolic arterial pressure DAP of the living body 14, e.g., the measurement end pressure value PCE preset to 60 mm Hg. Although for the compression pressure of the cuff 12 controlled in this manner, the upstream inflatable bladder 22, the intermediate inflatable bladder 24, and the downstream inflatable bladder 26 compress the living body 14 with the same compression pressure Pc, the compression pressure Pc of the cuff 12 detected by the fourth pressure sensor T4 is shown in FIG. 6.

Next, to obtain an real first pulse wave propagation velocity PWV1 and second pulse wave propagation velocity PWV2 as a plurality of pulse wave propagation velocities PWV of the living body 14 as the person to be measured, the compression pressure control portion 86 lowers the compression pressure Pc stepwise so as to form in sequence a first keep section (time point tk2 to time point tk3) where a constant first keep pressure PcH1 is kept temporarily and a second keep section (time point tk4 to time point tk5) where a second keep pressure PcH2 lower than the first keep pressure PcH1 is kept, to thereafter lower the pressure within each of the upstream inflatable bladder 22, the intermediate inflatable bladder 24, and the downstream inflatable bladder 26, to the atmospheric pressure. The first keep pressure PcH1 and the second keep pressure PcH2 are pressures sufficiently lower than the diastolic arterial pressure DAP of the living body 14 as the person to be measured, e.g., values previously set within a range of 20 to 60 mm Hg.

After generating the proper relationships expressed by e.g. Formulae (2) and (4) described later by the proper relationship generation portion 92 described later, in order to estimate an estimated systolic arterial pressure SAPe and an estimated diastolic arterial pressure DAPe of the living body 14 from Formulae (2) and (4), the compression pressure control portion 86 controls the compression pressure Pc so as to keep, in a monitor pressure keep section (time point tm2 to time point tm3), a pressure sufficiently lower than the diastolic arterial pressure DAP of the living body 14 as the person to be measured, e.g., a constant monitor pressure PcHm preset within the range of 20 to 60 mm Hg, in response to a blood pressure estimation start command (time point tm1) repeatedly issued from the electronic control device 70 at a predetermined blood pressure estimation cycle, e.g., a cycle of several tens of seconds to several minutes.

When the monitor pressure keep section (time point tm2 to time point tm3) comes to an end, the compression pressure control portion 86 lowers the pressure within each of the upstream inflatable bladder 22, the intermediate inflatable bladder 24, and downstream inflatable bladder 26, to the atmospheric pressure, by use of the quick exhaust valve 52. In response to the blood pressure estimation start command (time point tm1) repeatedly issued, the compression pressure control portion 86 repeatedly executes such a compression pressure cycle for blood pressure estimation. The monitor pressure PcHm may be equal to the first keep pressure PcH1 kept in the first keep section (time point tk2 to time point tk3) or the second keep pressure PcH2 kept in the second keep section (time point tk4 to time point tk5), or may be a keep pressure different therefrom.

Under a pressure sufficiently lower than the diastolic arterial pressure DAP of the living body 14 as the person to be measured, e.g., under the first keep pressure PcH1 of the first keep section preset within the range of 20 to 60 mm Hg, the pulse wave extraction portion 88 extracts and stores a pair of pulse waves MW11 and MW13 that are obtained, through a lowpass filter for pulse wave discrimination that discriminates signals in a wavelength band from 0 Hz to less than 25 Hz, from an output signal indicative of the compression pressure PcH1 of the upstream inflatable bladder 22 from the first pressure sensor T1 and from an output signal indicative of the compression pressure PcH1 of the downstream inflatable bladder 26 from the third pressure sensor T3.

Under the second keep pressure PcH2 of the second keep section set to a value lower than the first keep pressure PcH1, the pulse wave extraction portion 88 extracts a pair of pulse waves MW21 and MW23, through the lowpass filter for pulse wave discrimination, from an output signal indicative of the compression pressure PcH2 of the upstream inflatable bladder 22 from the first pressure sensor T1 and from an output signal indicative of the compression pressure PcH2 of the downstream inflatable bladder 26 from the third pressure sensor T3, and stores the pulse waves MW21 and MW23 extracted.

The pair of pulse waves MW11 and MW13 and the pair of pulse waves MW21 and MW23 are pressure oscillatory waves generated in synchronization with pulses superimposed on the compression pressures PcH1 and PcH2. The pulse wave extraction portion 88 stores the pulse waves MW11 and MW13, the pulse waves MW21 and MW23, and the compression pressures Pc at the time when the pulse waves occur, in association with one another. Since as described above, the pulse waves MW11 and MW13 and the pulse waves MW21 and MW23 are obtained by lowpass filter processing for pulse wave to discriminate signals in the wavelength band from 0 Hz to less than 25 Hz, the magnitudes of the pulse waves MW1 I and MW13 and the pulse waves MW21 and MW23 are represented by the same unit mm Hg as that of the compression pressure Pc, e.g., as shown in FIG. 16 described later.

The pulse wave propagation velocity calculation portion 90 calculates a time difference (propagation time) Δt113 between the pair of pulse waves MW11 and MW13 and a time difference (propagation time) Δt213 between the pair of pulse waves MW21 and MW23 obtained respectively in e.g. the first keep section (time point tk2 to time point tk3) and the second keep section (time point tk4 to time point tk5) that are a plurality of sections within a range where the compression pressure Pc of the cuff 12 is sufficiently lower than the diastolic arterial pressure DAP of the living body 14. The pulse wave propagation velocity calculation portion 90 then calculates and stores the pulse wave propagation velocity PWV1 (=L13/Δt113) of the first keep section and the pulse wave propagation velocity PWV2 (=L13/Δt213) of the second keep section, based on the time differences Δt113 and Δt213 and the length L13 as the propagation distance between the upstream inflatable bladder 22 and the downstream inflatable bladder 26.

FIG. 16 is a diagram indicating the amplitude of the pulse wave MW and its first derivative waveform dMW/dt that are superimposed in phase on a common time axis. FIG. 16 shows: that a zero-crossing point ZX1 from negative toward positive of the first derivative waveform dMW/dt of the pulse wave lies at the same time point as that of a local minimum site (local minimum point) MWLMP of the pulse wave MW; that a zero-crossing point ZX2 from positive toward negative of the first derivative waveform dMW/dt of the pulse wave lies at the same time point as that of a local maximum site (maximum peak point i.e. local maximum point) MWLXP of the pulse wave MW; and that a zero-crossing point ZX3 from negative toward positive of the first derivative waveform dMW/dt of the pulse wave lies at the same time point as that of a notch site (notch point i.e. dicrotic notch point) MWLNP posterior to the local maximum site of the pulse wave MW.

To generate Formula (2) expressing a proper relationship that estimates the estimated diastolic arterial pressure DAPe, the pulse wave propagation velocity calculation portion 90 calculates, as the time differences Δt113 and Δt213, respectively, a time difference Δt113_{D} between the local minimum sites of the pair of pulse waves MW11 and MW13 and a time difference Δt213_{D} between the local minimum sites of the pair of pulse waves MW21 and MW23. The local minimum sites of the pulse waves MW11 and MW13 and the local minimum sites of the pulse waves MW21 and MW23 can be, for example, rising points of the pulse waves MW11 and MW13 or zero-crossing points from negative toward positive of first derivative waves of the pulse waves MW11 and MW13, and rising points of the pulse waves MW21 and MW23 or zero-crossing points from negative toward positive of first derivative waves of the pulse waves MW21 and MW23. The pulse wave propagation velocity calculation portion 90 calculates a pulse wave propagation velocity PWV1_{D} (=L13/Δt113_{D}) of the first keep section and a pulse wave propagation velocity PWV2_{D} (=L13/Δt213_{D}) of the second keep section.

The pulse wave propagation velocity calculation portion 90 calculates a time difference Δt113_{S} between the local minimum sites of the pair of pulse waves MW11 and MW13 and a time difference Δt213_{S} between the local minimum sites of the pair of pulse waves MW21 and MW23 as the time differences Δt113 and Δt213 for use in generating Formula (4) expressing a proper relationship that estimates an estimated systolic arterial pressure SAPe. The local maximum sites of the pulse waves MW11 and MW13 and the local maximum sites of the pulse waves MW21 and MW23 can be, for example, maximum peak points of the pulse waves MW11 and MW13 or zero-crossing points from positive toward negative of the first derivative waves of the pulse waves MW11 and MW13, and maximum peak points of the pulse waves MW21 and MW23 or zero-crossing points from positive toward negative of the first derivative waves of the pulse waves MW21 and MW23. The pulse wave propagation velocity calculation portion 90 calculates a pulse wave propagation velocity PWV1_{S}(=L13/Δt113_{S}) of the first keep section and a pulse wave propagation velocity PWV2_{S}(=L13/Δt213_{S}) of the second keep section, for use in estimating the estimated systolic arterial pressure SAPe.

Although it is shown in FIG. 16 that the local minimum site MWLMP, the local maximum site MWLXP, the notch site MWLNP, etc. are obtained using the first derivative waveform dMW/dt of the pulse wave MW, they can be obtained using the pulse wave MW and its second derivative waveform d²MW/dt² as shown in FIG. 17. FIG. 17 is a diagram indicating the pulse wave MW and the second derivative waveform d²MW/dt² of the pulse wave MW in phase on a common time axis. FIG. 17 shows correspondence of the local minimum site MWLMP and the notch site MWLNP of the pulse wave MW with vertices ZT1 and ZT3 of the second derivative waveform of the pulse wave MW. In FIG. 17, the first vertex (peak point) ZT1 in a cycle of the second derivative waveform d²MW/dt² lies at the same time point as that of the local minimum site MWLMP that is the rising point of the pulse wave MW. The vertex ZT3 taking the maximum value on the second derivative waveform posterior to the time point ZT2 of the second derivative waveform lying at the same time point as that of the local maximum site MWLXP of the pulse wave MW lies at the same time point as that of the notch site MWLNP

In the case of using the second derivative waveform shown in FIG. 17, the pulse wave propagation velocity calculation portion 90 calculates, for example, as the time differences Δt113 and Δt213 for use in generating Formula (2) expressing the proper relationship that estimates the estimated diastolic arterial pressure DAPe, the time difference Δt113_{D} between vertices (peak points) ZT1 of second derivative waveforms of the pair of pulse waves MW11 and MW13 and the time difference Δt213_{D} between vertices (peak points) ZT1 of second derivative waveforms of the pair of pulse waves MW21 and MW23, respectively, and calculates the pulse wave propagation velocity PWV1_{D} (=L13/Δt113_{D}) of the first keep section and the pulse wave propagation velocity PWV2_{D}(=L13/Δt213_{D}) of the second keep section, respectively. Also in the case of generating Formula (6) expressing a proper relationship that estimates an estimated notch arterial pressure DNAPe, the pulse wave propagation velocity calculation portion 90 calculates time differences Δt113_{DN} and Δt213_{DN} and pulse wave propagation velocities PWV1_{DN} and PWV2_{DN}, in the same manner, from the second derivative waveforms.

After generating the proper relationships of formulae (2) and (4), the pulse wave propagation velocity calculation portion 90 calculates the time difference Δt113_{D} between the local minimum sites of the pair of pulse waves MW11 and MW13 and the time difference Δt113_{S} between the local maximum sites of the pair of pulse waves MW11 and MW13, in the monitor pressure keep section (time point tm2 to time point tm3) of the constant monitor pressure PcHm formed for each blood pressure estimation start command (time point tm1), and calculates, from those time differences Δt113_{D} and Δt113_{S}, a pulse wave propagation velocity PWV_{D} used for estimation of the estimated diastolic arterial pressure DAPe of Formula (2) and the pulse wave propagation velocity PWV_{S} used for estimation of the estimated systolic arterial pressure SAPe of Formula (4), respectively.

For the living body 14 as the person to be measured, the proper relationship generation portion 92 generates and stores each of the proper relationships expressed by formulae (2) and (4) between the real systolic arterial pressure SAP_{R} and the real diastolic arterial pressure DAP_{R}, and the real compression pressures in the low pressure section, i.e., the compression pressures PcH1 and PcH2 and the real pulse wave propagation velocities PWV1_{S} and PWV2_{S} or PWV1_{D} and PWV2_{D} obtained under the compression pressures PcH1 and PcH2. These proper relationships are repeatedly used for a subsequent monitoring cycle.

For the living body 14 as the person to be measured, the proper relationship generation portion 92 generates the proper relationship for the diastolic arterial pressure estimation expressed by Formula (2), by using, as the really measured calibration values, i_{D} and s_{D} that are respectively obtained as solutions to two unknowns i and s of two equations, each expressed by Formula (1) representing a linear relationship, when substituting, into each of the two equations, the diastolic arterial pressure DAP_{R} really measured by the blood pressure measurement portion 84 as the DAP and substituting thereinto PWV1_{D} and PWV2_{D} that are real pulse wave propagation velocities based on the time differences Δt113_{D} and Δt213_{D} between the local minimum sites of the pair of pulse waves obtained respectively for the plural compression pressures PcH1 (first keep pressure of the first keep section) and PcH2 (second keep pressure of the second keep section) within the low pressure section lower than the diastolic arterial pressure DAP of the living body 14 as the person to be measured.

DAPe=PWV_{D}/s_{D}−i_{D}/s_{D}+Pc (2)

For the living body 14 as the person to be measured, the proper relationship generation portion 92 generates the proper relationship for the systolic arterial pressure estimation expressed by Formula (4), by using, as the really measured calibration values, i_{S} and s_{S} that are respectively obtained as solutions to two unknowns i and s of two equations, each expressed by Formula (3) representing a linear relationship, when substituting, into each of the two equations, the diastolic arterial pressure DAP_{R} really measured by the blood pressure measurement portion 84 as the DAP and substituting thereinto PWV1_{S} and PWV2_{S} that are real pulse wave propagation velocities based on the time differences Δt113_{S} and Δt213_{S} between the local maximum sites of the pair of pulse waves obtained respectively for the plural compression pressures PcH1 (first keep pressure of the first keep section) and PcH2 (second keep pressure of the second keep section) within the low pressure section lower than the diastolic arterial pressure DAP of the living body 14 as the person to be measured.

SAPe=PWV_{S}²/s_{S}−i_{S}/s_{S}+Pc (4)

The blood pressure estimation portion 94 includes the diastolic arterial pressure estimation portion 96 and the systolic arterial pressure estimation portion 98. After finding the proper relationship expressed by Formula (2), the diastolic arterial pressure estimation portion 96 applies, for each blood pressure estimation cycle, the real compression pressure PcH1 in the low pressure section sufficiently lower than the diastolic arterial pressure DAP of the living body 14 and the real pulse wave propagation velocity PWV1_{D} obtained under the compression pressure PcH1 or the real compression pressure PcH2 and the real pulse wave propagation velocity PWV2_{D} obtained under the compression pressure PcH2, to the proper relationship expressed by Formula (2), to thereby estimate the estimated diastolic arterial pressure DAPe of the living body 14 as the person to be measured. For the compression pressure control, only one of the first keep section and the second keep section may be disposed. Estimated as the estimated diastolic arterial pressure DAPe may be a mean value of: an estimated diastolic arterial pressure obtained by applying the compression pressure PcH1 and the pulse wave propagation velocity PWV1_{D} to the proper relationship expressed by Formula (2); and a diastolic arterial pressure estimated by applying the compression pressure PcH2 and the pulse wave propagation velocity PWV2_{D} to the proper relationship expressed by Formula (2).

After finding the proper relationship expressed by Formula (4), the systolic arterial pressure estimation portion 98 applies, for each blood pressure estimation cycle, the real compression pressure PcH1 in the low pressure section sufficiently lower than the diastolic arterial pressure DAP of the living body 14 and the real pulse wave propagation velocity PWV1_{S} obtained under the compression pressure PcH1 or the real compression pressure PcH2 and the real pulse wave propagation velocity PWV2_{S} obtained under the compression pressure PcH2, to the proper relationship expressed by Formula (4), to thereby estimate the estimated systolic arterial pressure SAPe of the living body 14 as the person to be measured.

FIG. 18 shows a relationship between: the diastolic arterial pressures DAP_{R} each really measured using the intravascular catheter for blood pressure measurement at eight time points where blood pressure was extensively altered by drugs for one experimental animal (dog), conducted by the present inventors; and the estimated diastolic arterial pressures DAe each estimated by the diastolic arterial pressure estimation portion 96 using Formula (2) that is a proper relationship expression obtained as above by use of the blood pressure monitoring apparatus of this embodiment. Since the regression line of eight plots shown therein is given as Y=0.6648x+32.154 with a determination coefficient R² being R²=0.95, it was confirmed that a high correlation exists between the estimated diastolic arterial pressure DAPe estimated and the diastolic arterial pressure DAP_{R} really measured.

FIG. 19 is a flowchart explaining a principal part of control action of the electronic control device 70. When the blood pressure estimation start operation button 80 is turned on, the compression pressure Pc of the cuff 12 is raised at step (hereinafter, “step” will be omitted) S1 corresponding to the compression pressure control portion 86. Specifically, as shown in FIG. 6, the quick exhaust valve 52 is closed with the air pump 50 in operation so that compressed air pumped from the air pump 50 rapidly heightens the pressures within the main tube 56 and within the upstream inflatable bladder 22, intermediate inflatable bladder 24, and downstream inflatable bladder 26 communicating with the main tube 56. Compression on the upper arm 16 is then started by the cuff 12.

Next, at S2 corresponding to the compression pressure control portion 86, it is determined, based on an output signal of the fourth pressure sensor T4 indicative of the compression pressure Pc of the cuff 12, whether the compression pressure Pc is equal to or greater than a preset pressure-raise target pressure value PCM (e.g. 180 mm HG). At time points prior to time t2 of FIG. 6, determination at S2 is negative, and S1 and subsequent steps of FIG. 19 are repeatedly executed.

When the compression pressure Pc reaches the pressure-raise target pressure value PCM to allow determination at S2 to go affirmative, at S3 corresponding to the compression pressure control portion 86, the air pump 50 is deactivated and the exhaust control valve 54, the first on/off valve E1, the second on/off valve E2, the third on/off valve E3 are brought into operation so that gradual exhaust is made with a stepwise pressure lowering where the compression pressure Pc of the cuff 12 sequentially forms step pressures P1, P2, P3, . . . , Px preset e.g. every 3 to 5 mm Hg/sec. In the case of holding the step pressures P1, P2, P3, . . . , Px, the first on/off valve E1, the second on/off valve E2, and the third on/off valve E3 are each closed. Time t2 of FIG. 6 is a time point at which the gradual exhaust starts, and time t3 to time t4 is a time period in which the compression pressure Pc of the cuff 12 is held to the step pressure P1 for a predetermined time, e.g. for a time in which two beats occur.

Next, at step S4, while the compression pressures P1, P2, and P3 are each held for a predetermined time, output signals from the first pressure sensor T1, the second pressure sensor T2, and the third pressure sensor T3 are each subjected to lowpass filter processing for pulse wave sampling that discriminates signals in the wavelength band e.g. from 0 Hz to less than 25 Hz, to thereby extract pulse wave signals SM1, SM2, and SM3 indicative of pulse waves from the upstream inflatable bladder 22, the intermediate inflatable bladder 24, and the downstream inflatable bladder 26, whereas an output signal from the fourth pressure sensor T4 is subjected to lowpass filter processing for wavelength band of e.g. less than several Hz, to thereby extract and store the compression pressure Pc of the cuff 12 with AC components removed.

At S5 corresponding to the compression pressure control portion 86, it is determined whether the compression pressure Pc is equal to or less than a preset measurement end pressure value PCE (e.g. 60 mm Hg). In the case where this determination at S5 is negative, i.e., at time points anterior to time t11 of FIG. 6, the determination at S5 goes negative, so that S3 and subsequent steps are executed repeatedly.

If the determination at S5 is affirmative, at S6 and S7 corresponding to the blood pressure measurement portion 84, a pair of compression pressures Pc, corresponding respectively to inflection points of an envelope joining peak values of pulse wave signals SM2 (intermediate pulse waves) sequentially obtained in the process of the compression pressure Pc of the cuff 12 falling from a preset pressure-raise target pressure value PCM sufficiently higher than the systolic arterial pressure SAP, i.e., a local maximum point and a local minimum point of the first derivative waveform of the envelope, are measured as the real systolic arterial pressure SAP_{R} and diastolic arterial pressure DAP_{R}, respectively, of the living body 14 as the person to be measured. These real systolic arterial pressure SAP_{R} and diastolic arterial pressure DAP_{R} are used for generating the proper relationships i.e. Formulae (2) and (4) for use in blood pressure estimation of the living body 14 as the person to be measured.

Next, at S8 corresponding to the compression pressure control portion 86, control is performed so that the compression pressure Pc lies in the first keep section (time point tk2 to time point tk3) temporarily keeping the first keep pressure PcH1.

Subsequently, at S9 corresponding to the pulse wave extraction portion 88, a pair of pulse waves MW11 and MW13 are extracted, for storage, through a bandpass filter for pulse wave discrimination, from an output signal indicative of the compression pressure PcH1 of the upstream inflatable bladder 22 from the first pressure sensor T1 and an output signal indicative of the compression pressure PcH1 of the downstream inflatable bladder 26 from the third pressure sensor T3, respectively, under the first keep pressure PcH1.

Next, at S10 corresponding to the pulse wave propagation velocity calculation portion 90, the time difference Δt113_{D} between the local minimum sites of the pair of pulse waves MW11 and MW13 is calculated and the pulse wave propagation velocity PWV1_{D}(=L13/Δt113_{D}) of the first keep section is calculated from the time difference Δt113_{D}. Simultaneously, at S10, the time difference Δt113_{S} between the local maximum sites of the pair of pulse waves MW11 and MW13 is calculated and the pulse wave propagation velocity PWV1_{S}(=L13/Δt113_{S}) of the first keep section is calculated from the time difference Δt113_{S}.

Then, at S11 corresponding to the compression pressure control portion 86, control is performed so that the compression pressure Pc lies in the second keep section (time point tk4 to time point tk5) keeping the second keep pressure PcH2 lower than the first keep pressure PcH1.

Subsequently, at S12 corresponding to the pulse wave extraction portion 88, a pair of pulse waves MW21 and MW23 are extracted, for storage, through the bandpass filter for pulse wave discrimination, from an output signal indicative of the compression pressure PcH2 of the upstream inflatable bladder 22 from the first pressure sensor T1 and an output signal indicative of the compression pressure PcH2 of the downstream inflatable bladder 26 from the third pressure sensor T3, respectively, under the second keep pressure PcH2.

Next, at S13 corresponding to the pulse wave propagation velocity calculation portion 90, the time difference Δt213_{D} between the local minimum sites of the pair of pulse waves MW21 and MW23 is calculated and the pulse wave propagation velocity PWV2_{D} (=L13/Δt213_{D}) of the second keep section is calculated from the time difference Δt213_{D}. Simultaneously, at S13, the time difference Δt213_{S} between the local maximum sites of the pair of pulse waves MW21 and MW23 is calculated and the pulse wave propagation velocity PWV2_{S}(=L13/Δt213_{S}) of the second keep section is calculated from the time difference Δt213_{S}.

At S14 corresponding to the proper relationship generation portion 92, the proper relationship for the diastolic arterial pressure estimation expressed by Formula (2) is generated for the living body 14 as the person to be measured, by using, as the really measured calibration values, i_{D} and s_{D} that are respectively obtained as solutions to two unknowns i and s of two equations, each expressed by Formula (1) representing a linear relationship, when substituting the diastolic arterial pressure DAP_{R} really measured at S6 as the DAP into each of the two equations, and substituting thereinto PWV1_{D} and PWV2_{D} that are real pulse wave propagation velocities based on the time differences Δt113_{D} and Δt213_{D} between the local minimum sites of the pair of pulse waves obtained respectively for the first keep pressure PcH1 of the first keep section and the second keep pressure PcH2 of the second keep section.

At S14, the proper relationship for the systolic arterial pressure estimation expressed by Formula (4) is generated for the living body 14 as the person to be measured, by using, as the really measured calibration values, i_{S} and s_{S} that are respectively obtained as solutions to two unknowns i and s of two equations, each expressed by Formula (3) representing a linear relationship, when substituting the systolic arterial pressure SAP_{R} really measured at S7 as the SAP into each of the two equations, and substituting thereinto real pulse wave propagation velocities PWV1_{S} and PWV2_{S} as PWV based on the time differences Δt113_{S} and Δt213_{S} between the local minimum sites of the pair of pulse waves obtained respectively for the first keep pressure PcH1 of the first keep section and the second keep pressure PcH2 of the second keep section.

At succeeding S15, the quick exhaust valve 52 is activated so that the pressures within the upstream inflatable bladder 22, the intermediate inflatable bladder 24, and the downstream inflatable bladder 26 are each lowered to the atmospheric pressure.

At S16, it is determined whether the blood pressure estimation start command repeatedly issued at a predetermined blood pressure estimation cycle, e.g., a cycle of several tens of seconds to several minutes has been issued. If this determination at S16 is negative, the wait occurs, whereas if affirmative, a blood pressure estimation routine at S17 and subsequent steps is executed.

At S17 corresponding to the compression pressure control portion 86, control is performed so that the compression pressure Pc is raised up to a compression pressure of 20 to 60 mmHg lower than the diastolic arterial pressure DAP of the living body 14, for example, up to the monitor pressure PcHm, to form the monitor pressure keep section (time point tm2 to time point tm3) keeping the monitor pressure PcHm.

Subsequently, at S18 corresponding to the pulse wave extraction portion 88, a pair of pulse waves MWm1 and MWm3 are extracted, for storage, through the bandpass filter for pulse wave discrimination, from an output signal indicative of the compression pressure PcH1 of the upstream inflatable bladder 22 from the first pressure sensor T1 and an output signal indicative of the compression pressure PcH1 of the downstream inflatable bladder 26 from the third pressure sensor T3, respectively, under the monitor pressure PcHm of the monitor pressure keep section.

Next, at S19 corresponding to the pulse wave propagation velocity calculation portion 90, a time difference Δtm13_{D} between the local minimum sites of the pair of pulse waves MWm1 and MWm3 is calculated and a pulse wave propagation velocity PWVm_{D}(=L13/Δtm13_{D}) of the monitor pressure keep section is calculated from the time difference Δtm13_{D}. A time difference Δtm13_{S} between the local maximum sites of the pair of pulse waves MWm1 and MWm3 is calculated and a pulse wave propagation velocity PWVm_{S} (=L13/Δtm13_{S}) of the monitor pressure keep section is calculated from the time difference Δtm13_{S}.

Then, at S20 corresponding to the diastolic arterial pressure estimation portion 96, the estimated diastolic arterial pressure DAPe is calculated by applying the monitor pressure PcHm and the pulse wave propagation velocity PWVm_{D} to Formula (2) expressing the proper relationship of the living body 14 as a target to be measured. At S21 corresponding to the systolic arterial pressure estimation portion 98, the estimated systolic arterial pressure SAPe is calculated by applying the monitor pressure PcHm and the pulse wave propagation velocity PWVm_{S} to Formula (4) expressing the proper relationship of the living body 14 as the target to be measured.

At succeeding S22, the estimated diastolic arterial pressure DAPe and the estimated systolic arterial pressure SAPe estimated are stored, and displayed on the display device 78. At succeeding S23, the pressures within the upstream inflatable bladder 22, the intermediate inflatable bladder 24, and the downstream inflatable bladder 26 are each lowered to the atmospheric pressure. It is then determined at S24 whether a stop (off) operation by the blood pressure estimation start operation button 8 has been made. While determination at S24 continues to be negative, the blood pressure estimation routine at S16 and subsequent steps are repeated and, if determination at S24 goes affirmative, the blood pressure monitoring routine is brought to an end.

As above, the proper relationship generation portion 92 generates the proper relationships (Formulae (2) and (4)) of the living body 14 between: the estimated arterial pressures APe (DAPe and SAPe); and the real compression pressures PcH1 and PcH2, the real pulse wave propagation velocities PWV1 (PWV1_{D} and PWV1_{S}), and pulse wave propagation velocities PWV2 (PWV2_{D} and PWV2_{S}), by applying: the real arterial pressures AP_{R} (DAP_{R} and SAP_{R}) of the living body 14; and the pulse wave propagation velocity PWV1 (PWV1_{D} and PWV1_{S})under the real compression pressure PcH1 in the low pressure section lower than the diastolic arterial pressure DAP of the living body 14 and the pulse wave propagation velocity PWV2 (PWV2_{D} and PWV2_{S}) under the real compression pressure PcH2, to the regression lines given by the previously stored linear relationships (Formulae (1) and (3)) between the transmural pressure (AP−Pc) of the artery 18 and the squared values PWV² of the plurality of pulse wave propagation velocities respectively detected under the plurality of compression pressures in the low pressure section lower than the diastolic arterial pressure DAP of the living body 14 by the cuff 12, whereas the blood pressure estimation portion 94 estimates the estimated arterial pressures APe (SAPe and DAPe) of the living body 14 by applying the real compression pressure PcHm and the real pulse wave propagation velocities PWVm (PWVm_{D} and PWVm_{S}) to the proper relationships (Formulae (2) and (4)).

As described above, the blood pressure monitoring apparatus 10 of this embodiment is the blood pressure monitoring apparatus 10 having the cuff 12 that has the plurality of inflatable bladders 22, 24, and 26 forming independent air chambers juxtaposed in the width direction and that is wrapped around the upper arm (site to be compressed) 16 of the living body 14 (person to be measured) to compress the artery 18 of the living body 14, the blood pressure monitoring apparatus 10 repeatedly estimating the estimated arterial pressure APe of the living body 14, the blood pressure monitoring apparatus 10 including: the linear relationship storage portion 82 that stores previously stored linear relationships between the plurality of transmural pressures of the artery 18 that are pressure differences between the arterial pressures AP within the artery 18 and the plurality of compression pressures Pc of the cuff 12, and the squared values PWV² of the pulse wave propagation velocities respectively detected under the plurality of compression pressures Pc of the cuff 12 in the low pressure section lower than the diastolic arterial pressure DAP of the living body 14; the blood pressure measurement portion 84 that measures the real arterial pressure AP_{R} of the living body 14, based on the pulse synchronous wave from the artery 18 obtained in the pressure lowering process after compressing the upper arm 16 of the living body 14 with the compression pressure Pc higher than the systolic arterial pressure SAP of the living body 14; the proper relationship generation portion 92 that generates the proper relationship on the living body 14 among the real arterial pressure AP_{R} of the living body 14, the real compression pressures PcH1 and PcH2, and the real pulse wave propagation velocities PWV1 and PWV2, by applying, for the living body 14, the real arterial pressure AP_{R}, the plurality of real compression pressures PcH1 and PcH2 in the low pressure section, and the real pulse wave propagation velocities PWV1 and PWV2 based on the propagation time between the pulse waves obtained respectively under the real compression pressures PcH1 and PcH2, to the linear relationships; and the blood pressure estimation portion 94 that estimates the estimated arterial pressure APe of the living body 14 by applying the real compression pressure PcHm in the low pressure section and the real pulse wave propagation velocity PWVm obtained with the real compression pressure PcHm, to the proper relationship on the living body 14. In consequence, except when the blood pressure measurement portion 84 measures the real systolic arterial pressure SAP_{R} and the real diastolic arterial pressure DAP_{R} of the living body 14, the compression pressure Pc of the cuff 12 can be a lower value than the diastolic arterial pressure DAP of the living body 14, enabling application of the compression pressure PcHm in a shortened time (for several seconds) and blood pressure measurement at short intervals, thus rendering it possible to alleviate the burden on the living body 14 and continuously estimate the blood pressure fluctuation in a shorter time.

According to the blood pressure monitoring apparatus 10 of this embodiment, the proper relationship generation portion 92 uses the real diastolic arterial pressure DAP_{R} of the living body 14, the plurality of real compression pressures (first keep pressure PcH1 and second keep pressure PcH2), and the pulse wave propagation velocities (PWV1_{D} and PWV2_{D}) based on the time differences Δt113_{D} and Δt213_{D} between the local minimum sites of the pulse waves obtained respectively under the plurality of real compression pressures, to generate Formula (2) that is a proper relationship expression of the living body 14 among the estimated diastolic arterial pressure DAPe, the plurality of compression pressures (first keep pressure PcH1 and second keep pressure PcH2), and the pulse wave propagation velocities (PWV1_{D} and PWV2_{D}), whereupon the diastolic arterial pressure estimation portion 96 can easily estimate the estimated diastolic arterial pressure DAPe of the living body 14 by applying the real compression pressure (e.g. first keep pressure PcH1) obtained in the low pressure section lower than the diastolic arterial pressure DAP and the pulse wave propagation velocity PWV1_{D} based on the time difference Δt113_{D} between the local minimum sites of the pulse waves obtained under the real compression pressure, to the proper relationship of Formula (2) generated by the proper relationship generation portion 92.

According to the blood pressure monitoring apparatus 10 of this embodiment, the time difference (propagation time) Δt113_{D} between the local minimum sites of the pair of pulse waves MW11 and MW13 is a propagation time between the respective rising points of the pulse waves MW11 and MW13. This makes it easy to obtain the time difference Δt113_{D} between the local minimum sites of the pair of pulse waves MW11 and MW13, leading to enhanced blood pressure estimation accuracy.

According to the blood pressure monitoring apparatus 10 of this embodiment, the blood pressure estimation portion 94 includes the diastolic arterial pressure estimation portion 96 that estimates the estimated diastolic arterial pressure DAPe of the living body 14 by successively applying, for the living body 14 as the person to be measured, the real compression pressure PcH1 or PcH2 in the low pressure section lower than the diastolic arterial pressure DAP of the living body 14 and the real pulse wave propagation velocity PWV1_{D} or PWV2_{D} obtained under the real compression pressure PcH1 or PcH2, to the proper relationship of Formula (2). This can alleviate the burden on the living body 14, enabling easy estimation of the estimated diastolic arterial pressure DAPe of the living body 14.

According to the blood pressure monitoring apparatus 10 of this embodiment, the proper relationship generation portion 92 uses the real systolic arterial pressure SAP_{R} of the living body 14, the plurality of real compression pressures (first keep pressure PcH1 and second keep pressure PcH2), and the pulse wave propagation velocities (PWV1_{S} and PWV2_{S}) based on the time differences Δt113_{S} and Δt213_{S} between the local maximum sites of the pulse waves obtained respectively under the plurality of real compression pressures, to generate Formula (4) that is a proper relationship expression of the living body 14 among the estimated systolic arterial pressure SAPe, the compression pressures, and the pulse wave propagation velocities, whereupon the systolic arterial pressure estimation portion 98 can estimate the estimated systolic arterial pressure SAPe of the living body 14 by applying the real compression pressure (e.g. first keep pressure PcH1) obtained in the low pressure section lower than the diastolic arterial pressure DAP and the pulse wave propagation velocity PWV1_{S} based on the time difference Δt113_{S} between the local maximum sites of the pulse waves obtained under the real compression pressure, to Formula (4) generated by the proper relationship generation portion 92.

According to the blood pressure monitoring apparatus 10 of this embodiment, the time difference (propagation time) Δt113_{S} between the local maximum sites of the pair of pulse waves MW11 and MW13 is a propagation time between the local maximum points of the pulse waves MW11 and MW13. This makes it easy to obtain the propagation time between the local maximum sites of the pulse waves, leading to enhanced blood pressure estimation accuracy.

According to the blood pressure monitoring apparatus 10 of this embodiment, the blood pressure estimation portion 94 includes the systolic arterial pressure estimation portion 98 that estimates the estimated systolic arterial pressure SAPe of the living body 14 by successively applying, for the living body 14 as the person to be measured, the real compression pressure PcH1 or PcH2 in the low pressure section lower than the diastolic arterial pressure DAP of the living body 14 and the real pulse wave propagation velocity PWV1_{S} or PWV2_{S} obtained under the real compression pressure PcH1 or PcH2, to the proper relationship of Formula (4). This can alleviate the burden on the living body 14, enabling easy estimation of the estimated systolic arterial pressure SAPe of the living body 14.

The blood pressure monitoring apparatus 10 of this embodiment includes: the compression pressure control portion 86 that lowers stepwise the plurality of compression pressures (first keep pressure PcH1 and second keep pressure PcH2) lying in the low pressure section lower than the diastolic arterial pressure DAP of the living body 14 so as to form, in the low pressure section lower than the diastolic arterial pressure DAP of the living body 14, the plurality of sections (first keep section and second keep section) where the compression pressures are temporarily kept at constant values; the pulse wave extraction portion 88 that extracts pulse waves as pressure oscillations generated in synchronization with pulses within the plurality of inflatable bladders (upstream inflatable bladder 22 and downstream inflatable bladder 26), respectively, under the compression pressures in the plurality of sections; and the pulse wave propagation velocity calculation portion 90 that calculates the pulse wave propagation velocity, based on the time difference between pulse waves obtained in each of the plurality of sections and the length (L13) between the plurality of inflatable bladders. Hence, the pulse waves obtained in each of the sections (first keep section and second keep section) where the compression pressures are kept at constant values have waveforms without distortion caused by fluctuations of the compression pressure, ensuring correct obtainment of the pulse wave propagation velocity PWV and correct calculation of Formulae (2) and (4) that are the proper relationship expressions of the living body 14.

According to the blood pressure monitoring apparatus 10 of this embodiment, the cuff 12 is wrapped around the site to be compressed of the living body 14 and has the independent upstream inflatable bladder 22, intermediate inflatable bladder 24, and downstream inflatable bladder 26 juxtaposed across the width and each compressing the site to be compressed of the living body 14, the upstream inflatable bladder 22, intermediate inflatable bladder 24, and downstream inflatable bladder 26 each compressing the artery 18 within the site to be compressed at the same compression pressure. This is advantageous in that the blood pressure measurement using compression on the four limbs of the living body 14 and the detection of the pulse wave propagation velocity PWV can be performed at the same time.

### Second Embodiment

A blood pressure monitoring apparatus 110 of another embodiment of the present invention will then be described. In the following, portions common to the above embodiment are designated by the same reference numerals and explanations thereof will be omitted.

In the above embodiment, to estimate the estimated systolic arterial pressure SAPe of the living body 14, the proper relationship generation portion 92 generates Formula (4) that is a proper relationship expression of the person to be measured among the estimated systolic arterial pressure SAPe, the compression pressure, and the pulse wave propagation velocity, by using the real systolic arterial pressure SAP_{R} of the living body 14, the plurality of real compression pressures (first keep pressure PcH1 and second keep pressure PcH2), and the pulse wave propagation velocities (PWV1_{S} and PWV2_{S}) based on the time differences Δt113_{S} and Δt213_{S} between the local maximum sites of the pulse waves obtained under the plurality of real compression pressures, whereas the systolic arterial pressure estimation portion 98 estimates the estimated systolic arterial pressure SAPe of the living body 14 by applying to Formula (4) generated by the proper relationship generation portion 92 the real compression pressure (e.g. first keep pressure PcH1) obtained in the low pressure section lower than the diastolic arterial pressure DAP and the pulse wave propagation velocity PWV1_{S} based on the time difference Δt113_{S} between the local maximum sites of the pulse waves obtained under the real compression pressure. On the other hand, this embodiment differs in that it uses the estimation method similar to the above to estimate the estimated notch arterial pressure DNAPe that is a blood pressure at the time of occurrence of a notch site locally formed posterior to the local maximum site, to estimate the estimated systolic arterial pressure SAPe from the estimated notch arterial pressure DNAPe.

FIG. 20 is a function block diagram explaining control function of an electronic control device 170 of this embodiment. Similarly to the linear relationship storage portion 82, a linear relationship storage portion 182 stores a regression line representing a linear relationship given by Formula (5), for a notch arterial pressure DNAP, in addition to the stored linear relationships of Formulae (1) and (3) between the squared values PWV² of the plurality of pulse wave propagation velocities PWV respectively detected under the plurality of compression pressures Pc of the cuff 12 in the low pressure section lower than the diastolic arterial pressure DAP of the living body 14, and the transmural pressure (AP−Pc) of the artery 18 that is the pressure difference between the arterial pressure AP within the artery 18 and the compression pressures Pc. This regression line given by Formula (5) is derived from Bramwell Hill's formula (7), similarly to the above first embodiment, by way of Formulae (8) to (14). It should be noted that the pulse wave propagation velocity PWV of Formula (5) is obtained from the time difference Δt between positions of notch sites MWLNP of a pair of pulse waves obtained respectively from the upstream inflatable bladder 22 and the downstream inflatable bladder 26 in a constant pressure period within the pressure range lower than the diastolic arterial pressure DAP of the living body 14. The positions of these notch sites MWLNP are obtained from the first derivative waveforms of the pulse waves MW and the second derivative waveforms of the pulse waves MW, as shown in FIGS. 16 and 17 described above.

PWV²=s·(DNAP−Pc)+i (5)
where s denotes a slope of the regression line and i denotes an intercept of the regression line.

FIG. 21 is a diagram showing, together with the regression line y and the determination coefficient R², two-dimensional coordinate data indicative of results of Experiment No. 9 performed by the present inventors for the relationship between the transmural pressure (DNAP−Pc) and the squared value PWV² of the pulse wave propagation velocity on a predetermined living body 14. The determination coefficient R² in these results is 0.9779 approximate to 1, and therefore the regression line expressing high-quality linear relationship was obtained.

Similarly to the blood pressure measurement portion 84, a blood pressure measurement portion 184 measures the real diastolic arterial pressure DAP_{R} of the living body 14 as the person to be measured using a blood pressure measuring apparatus, prior to generation of the proper relationship of Formula (6) described later by a proper relationship generation portion 192. The blood pressure measurement portion 184 measures a mean arterial pressure MAP of the living body 14 using the blood pressure measuring apparatus and determines the measured mean arterial pressure MAP as a real notch arterial pressure DNAP_{R} of the living body 14. The mean arterial pressure MAP is a compression pressure Pc at the time when the pulse wave indicates its maximum amplitude, and in the automatic blood pressure measuring apparatus e.g. of oscillometric type, measured as the mean arterial pressure MAP is a compression pressure Pc at the point time indicating a maximum value (maximum peak value) of an envelope joining peak values of pulse wave signals SM2 (intermediate pulse waves) obtained in sequence in the process of the compression pressure Pc of the cuff 12 being lowered from the preset pressure-raise target pressure value PCM sufficiently higher than the systolic arterial pressure SAP. The mean arterial pressure MAP measured in this manner is approximate to and substantially equivalent to the notch arterial pressure DNAP of the living body 14. FIG. 22 shows the results of the experiment performed by the present inventors and shows the correlation on an animal (dog) between the notch arterial pressure DNAP directly measured using a catheter and the mean arterial pressure MAP really measured.

Similarly to the compression pressure control portion 86, a compression pressure control portion 186 executes compression pressure control for blood pressure measurement, as shown in a section from time point t1 to time point t11 of FIG. 6, and then performs compression pressure control shown in a section between time point tk1 and time point tk5 to generate proper relationship of Formula (6). Then, to estimate the estimated systolic arterial pressure SAPe from the estimated notch arterial pressure DNAPe and the estimated diastolic arterial pressure DAPe of the living body 14, the blood pressure measurement portion 184 controls the compression pressure Pc so as to form a constant monitor pressure PcHm shown in the monitor pressure keep section from time point tm1 to time point tm3 of FIG. 6, in response to the blood pressure estimation start command (time point tm1) issued repeatedly at a predetermined blood pressure estimation cycle.

Similarly to the pulse wave extraction portion 88, a pulse wave extraction portion 188 extracts, for storage, the pair of pulse waves MW11 and MW13 from the pulse wave signals SM1 and SM3, respectively, obtained through the lowpass filter for pulse wave discrimination that discriminates signals in the wavelength band of 0 Hz to less than 25 Hz, from an output signal indicative of the compression pressure PcH1 of the upstream inflatable bladder 22 from the first pressure sensor T1 and an output signal indicative of the compression pressure PcH1 of the downstream inflatable bladder 26 from the third pressure sensor T3, within the range of e.g. 20 to 60 mmHg that is a pressure sufficiently lower than the diastolic arterial pressure DAP of the living body 14 as the person to be measured. Alternatively, the pulse wave extraction portion 188 extracts, for storage, the pair of pulse waves MW21 and MW23 from the pair of upstream inflatable bladder 22 and downstream inflatable bladder 26, through the lowpass filter for pulse wave discrimination that discriminates signals in the wavelength band less than 25 Hz, from an output signal indicative of the compression pressure PcH2 of the upstream inflatable bladder 22 from the first pressure sensor T1 and an output signal indicative of the compression pressure PcH2 of the downstream inflatable bladder 26 from the third pressure sensor T3, under the second keep pressure PcH2 of the second keep section set to a value lower than the first keep pressure PcH1.

To generate the proper relationship of Formula (2) between the diastolic arterial pressure DAP and the pulse wave propagation velocity on the predetermined living body 14, similarly to the pulse wave propagation velocity calculation portion 90, a pulse wave propagation velocity calculation portion 190 calculates a time difference Δt113_{D} between the local minimum sites of the pair of pulse waves MW11 and MW13 extracted in the first keep section (time point tk2 to time point tk3), to calculate a pulse wave propagation velocity PWV1_{D} (=L13/Δt113_{D}) of the first keep section and calculates a time difference Δt213_{D} between the local minimum sites of the pair of pulse waves MW21 and MW23 extracted in the second keep section (time point tk4 to time point tk5), to calculate a pulse wave propagation velocity PWV2_{D} (=L13/Δt213_{D}) of the second keep section, for storage.

To generate the proper relationship of Formula (6) between the notch arterial pressure DNAP and the pulse wave propagation velocity PWV on the predetermined living body 14, the pulse wave propagation velocity calculation portion 190 calculates and stores the time difference Δt113_{DN} between the notch sites of the pair of pulse waves MW11 and MW13 extracted in the first keep section (time point tk2 to time point tk3), to calculate the pulse wave propagation velocity PWV1_{DN} (=L13/Δt113_{DN}) of the first keep section and calculates a time difference Δt213_{DN} between the notch sites of the pair of pulse waves MW21 and MW23 extracted in the second keep section (time point tk4 to time point tk5), to calculate, for storage, a pulse wave propagation velocity PWV2_{DN} (L13/Δt213_{DN}) of the second keep section.

After generating the proper relationships of Formulae (2) and (6), the pulse wave propagation velocity calculation portion 190 makes calculation based on the time difference Δt113_{D} between the local minimum sites and the time difference Δt113_{DN} between the notch sites, of the pair of pulse waves MW11 and MW13, in the monitor pressure keep section (time point tm2 to time point tm3) of the constant monitor pressure PcHm formed for each blood pressure estimation start command (time point tm1), to calculate, for storage, the pulse wave propagation velocity PWV_{D} for use in estimating the estimated diastolic arterial pressure DAPe using Formula (2) and a pulse wave propagation velocity PWV_{DN} for use in estimating the estimated notch arterial pressure DNAPe using Formula (6).

Similarly to the proper relationship generation portion 92 of the above first embodiment, the proper relationship generation portion 192 generates and stores, for the living body 14 as the person to be measured, each of the proper relationships expressed by Formula (2) among the real diastolic arterial pressure DAP_{R}, the real monitor pressures i.e. the compression pressures PcH1 and PcH2 in the low pressure section, and the real pulse wave propagation velocities PWV1_{D} and PWV2_{D} obtained under the compression pressures PcH1 and PcH2. Then, the proper relationship generation portion 192 generates and stores each of the proper relationships expressed by Formula (6) among the real notch arterial pressure DNAP_{R}, the real compression pressures i.e. the compression pressures PcH1 and PcH2 in the low pressure section, and the real pulse wave propagation velocities PWV1_{DN} and PWV2_{DN} obtained under the compression pressures PcH1 and PcH2.

DNAPe=PWV_{DN}²/s_{DN}−i_{DN}/s_{DN}+Pc (6)

For the living body 14 as the person to be measured, the proper relationship generation portion 192 generates the proper relationship for notch arterial pressure estimation expressed by Formula (6), by using, as the really measured calibration values, i_{DN} and s_{DN} that are respectively obtained as solutions to two unknowns i and s of two equations, each expressed by Formula (5) representing a linear relationship, when substituting, into each of the two equations, the notch arterial pressure DNAP_{R} really measured by the blood pressure measurement portion 184 as the DNAP and substituting thereinto PWV1_{DN} and PWV2_{DN} that are real pulse wave propagation velocities based on the time differences Δt113_{DN} and Δt213_{DN} between the notch sites of the pair of pulse waves obtained respectively for the plural compression pressures Pch1 (first keep pressure of the first keep section) and PcH2 (second keep pressure of the second keep section) within the low pressure section lower than the diastolic arterial pressure DAP of the living body 14 as the person to be measured.

A blood pressure estimation portion 194 includes a diastolic arterial pressure estimation portion 196, a notch arterial pressure estimation portion 200, and a systolic arterial pressure estimation portion 198. After finding the proper relationship expressed by Formula (2), the diastolic arterial pressure estimation portion 196 applies, for each blood pressure estimation cycle, the real compression pressure PcH1 in the low pressure section sufficiently lower than the diastolic arterial pressure DAP of the living body 14 and the real pulse wave propagation velocity PWV1_{D} obtained under the compression pressure PcH1 or the real compression pressure PcH2 and the real pulse wave propagation velocity PWV2_{D} obtained under the compression pressure PcH2, to the proper relationship expressed by Formula (2), to thereby estimate the estimated diastolic arterial pressure DAPe of the living body 14 as the person to be measured.

After finding the proper relationship expressed by Formula (6), the notch arterial pressure estimation portion 200 applies, for each blood pressure estimation cycle, the real compression pressure PcH1 in the low pressure section sufficiently lower than the diastolic arterial pressure DAP of the living body 14 and the real pulse wave propagation velocity PWV1_{DN} obtained under the compression pressure PcH1 or the real compression pressure PcH2 and the real pulse wave propagation velocity PWV2_{DN} obtained under the compression pressure PcH2, to the proper relationship expressed by Formula (6), to thereby estimate the estimated notch arterial pressure DNAPe of the living body 14 as the person to be measured.

Since the magnitude of the pulse wave MW obtained at a compression pressure lower than the diastolic arterial pressure DAP of the living body 14, e.g. at the monitor pressure PcHm has the same unit (mm Hg) as that of the compression pressure Pc, the systolic arterial pressure estimation portion 198 utilizes that as shown in FIG. 23, the local minimum site, local maximum site, and notch site of the pulse wave MW correspond respectively to the diastolic arterial pressure DAP, the systolic arterial pressure SAP, and the notch arterial pressure DNAP, the systolic arterial pressure estimation portion 198, to generate a relationship shown in FIG. 24, based on the estimated diastolic arterial pressure DAPe estimated by the diastolic arterial pressure estimation portion 196, the estimated notch arterial pressure DNAPe estimated by the notch arterial pressure estimation portion 200, the real compression pressure Pc at the local minimum site of the pulse wave MW of the living body 14 as the target to be measured, and the compression pressure Pc at the notch site.

Then, from the relationship shown in FIG. 24, the systolic arterial pressure estimation portion 198 estimates the estimated systolic arterial pressure SAPe, based on the compression pressure (cuff pressure) Pc indicative of the real magnitude at the local maximum site of the pulse wave MW obtained at the monitor pressure PcHm from the living body 14 as the target to be measured. FIG. 24 shows that the estimated systolic arterial pressure SAPe estimated when the real magnitude at the local maximum site of the pulse wave MW was 55.2 mm Hg was 115 mm Hg. Although in FIG. 24 the estimated systolic arterial pressure SAPe is estimated assuming a linear relationship between the estimated diastolic arterial pressure DAPe/the estimated notch arterial pressure DNAPe and the compression pressure Pc corresponding thereto, a non-linear relationship such as an exponential function may be assumed and used.

FIG. 25 is a flowchart explaining a principal part of control action of the electronic control device 170 of this embodiment. In the following, differences from FIG. 19 will mainly be described.

S31 to S36 are similar to S1 to S6 of FIG. 19. At S37 corresponding to the blood pressure measurement portion 184, the notch arterial pressure DNAP_{R} is measured. In the automatic blood pressure measuring apparatus e.g. of oscillometric type, measured as the mean arterial pressure MAP is a compression pressure Pc at the point time indicating a maximum value (maximum peak value) of an envelope joining peak values of pulse wave signals SM2 (intermediate pulse waves) obtained in sequence in the process of the compression pressure Pc of the cuff 12 being lowered from the preset pressure-raise target pressure value PCM sufficiently higher than the systolic arterial pressure SAP.

At succeeding S38 corresponding to the compression pressure control portion 186, similarly to S8 of FIG. 19, the first keep pressure PcH1 is kept, and at S39 corresponding to the pulse wave extraction portion 188, similarly to S9 of FIG. 19, the pulse wave is extracted at the first keep pressure PcH1.

At S40 corresponding to the pulse wave propagation velocity calculation portion 190, the pulse wave propagation velocity PWV1_{D} and the pulse wave propagation velocity PWV1_{DN} at the first keep pressure PcH1 are calculated. The pulse wave propagation velocity PWV1_{D} serves to generate the proper relationship of Formula (2) between the diastolic arterial pressure DAP and the pulse wave propagation velocity PWV on the predetermined living body 14 and is the pulse wave propagation velocity PWV1_{D} (=L13/Δt113_{D}) of the first keep section (time point tk2 to time point tk3), calculated from the time difference Δt113_{D} between the local minimum sites of the pair of pulse waves MW11 and MW13 extracted at the first keep section. The pulse wave propagation velocity PWV1_{DN} is used to generate Formula (6) that is the proper relationship expression of the living body 14 as the target to be measured and is the pulse wave propagation velocity PWV1_{DN} (=L13/Δt113_{DN}) of the first keep section, calculated from the time difference Δt113_{DN} between the notch sites of the pair of pulse waves MW11 and MW13 extracted at the first keep pressure PcH1.

At succeeding S41 corresponding to the compression pressure control portion 186, similarly to S11 of FIG. 19, the second keep pressure PcH2 is kept, and at S42 corresponding to the pulse wave extraction portion 188, similarly to S12 of FIG. 19, the pulse wave is extracted at the second keep pressure PcH2.

At S43 corresponding to the pulse wave propagation velocity calculation portion 190, the pulse wave propagation velocity PWV2_{D} and the pulse wave propagation velocity PWV2_{DN} at the second keep pressure PcH2 are calculated. The pulse wave propagation velocity PWV2_{D} serves to generate the proper relationship of Formula (2) between the diastolic arterial pressure DAP and the pulse wave propagation velocity PWV on the predetermined living body 14 and is the pulse wave propagation velocity PWV2_{D} (=L13/Δt213_{D}) of the second keep section (time point tk4 to time point tk5), calculated from the time difference Δt213_{D} between the local minimum sites of the pair of pulse waves MW21 and MW23 extracted at the second keep section. The pulse wave propagation velocity PWV2_{DN} is used to generate Formula (6) that is the proper relationship expression of the living body 14 as the target to be measured and is the pulse wave propagation velocity PWV2_{DN} (=L13/Δt213_{DN}) of the second keep section, calculated from the time difference Δt213_{DN} between the notch sites of the pair of pulse waves MW21 and MW23 extracted at the second keep pressure PcH2.

At S44 corresponding to the proper relationship generation portion 192, the proper relationship for the diastolic arterial pressure estimation expressed by Formula (2) is generated for the living body 14 as the person to be measured, by using, as the really measured calibration values, i_{D} and S_{D} that are respectively obtained as solutions to two unknowns i and s of two equations, each expressed by Formula (1) representing a linear relationship, when substituting the diastolic arterial pressure DAP_{R} really measured at S36 into each of the two equations, and substituting thereinto the real pulse wave propagation velocities PWV1_{D} and PWV2_{D} based on the time differences Δt113_{D} and Δt213_{D} between the local minimum sites of the pair of pulse waves obtained respectively for the first keep pressure PcH1 of the first keep section and the second keep pressure PcH2 of the second keep section.

At S44, the proper relationship for the notch arterial pressure estimation expressed by Formula (6) is generated for the living body 14 as the person to be measured, by using, as the really measured calibration values, i_{DN} and s_{DN} that are respectively obtained as solutions to two unknowns i and s of two equations, each expressed by Formula (5) representing a linear relationship, when substituting the notch arterial pressure DNAP_{R} really measured at S37 into each of the two equations, and substituting thereinto the real pulse wave propagation velocities PWV1_{DN} and PWV2_{DN} based on the time differences Δt113_{DN} and Δt213_{DN} between the notch sites of the pair of pulse waves obtained respectively for the first keep pressure PcH1 of the first keep section and the second keep pressure PcH2 of the second keep section.

At succeeding S45, similarly to S15, the quick exhaust valve 52 is activated so that the pressures within the upstream inflatable bladder 22, the intermediate inflatable bladder 24, and the downstream inflatable bladder 26 are each pumped down to the atmospheric pressure.

At S46 to S48, similarly to S16 to S18 of FIG. 19, control is performed so that when a blood pressure estimation start command is issued, the compression pressure Pc is raised up to a compression pressure of 20 to 60 mm Hg, e.g., the monitor pressure PcHm to form the monitor pressure keep section (time point 2 to time point 3) keeping the monitor pressure PcHm, and the pair of pulse waves MWm1 and MWm3 are extracted, through the bandpass filter for pulse wave discrimination, from an output signal indicative of the compression pressure PcHm of the upstream inflatable bladder 22 from the first pressure sensor T1 and an output signal indicative of the compression pressure PcHm of the downstream inflatable bladder 26 from the third pressure sensor T3, respectively, under the monitor pressure PcHm of the monitor pressure keep section.

Next, at S49 corresponding to the pulse wave propagation velocity calculation portion 190, the time difference Δtm13_{D} between the local minimum sites of the pair of pulse waves MWm1 and MWm3 is calculated, and the pulse wave propagation velocity PWVm_{D} (=L13/Δtm13_{D}) at the monitor pressure keep section is calculated from the time difference Δtm13_{D}. A time difference Δtm13_{DN} between the notch sites of the pair of pulse waves MWm1 and MWm3 is calculated, and the pulse wave propagation velocity PWVm_{DN} (=L13/Δtm13_{DN}) at the monitor pressure keep section is calculated from the time difference Δtm13_{DN}.

At S50 corresponding to the diastolic arterial pressure estimation portion 196, the estimated diastolic arterial pressure DAPe is calculated by applying the monitor pressure PcHm and the pulse wave propagation velocity PWVm_{D} to Formula (2) expressing a proper relationship of the living body 14 as the target to be measured. At S51 corresponding to the notch arterial pressure estimation portion 200, the estimated notch arterial pressure DNAPe is calculated by applying the monitor pressure PcHm and the pulse wave propagation velocity PWVm_{DN} to Formula (6) expressing a proper relationship of the living body 14 as the target to be measured.

Then, at S52 corresponding to the systolic arterial pressure estimation portion 198, the relationship shown in FIG. 24 is generated based on the estimated diastolic arterial pressure DAPe estimated at S50, the estimated notch arterial pressure DNAPe estimated at S51, and the real compression pressures Pc at the local minimum site and the notch site of the pulse wave MW of the living body 14 as the target to be measured. Next, at S52, the estimated systolic arterial pressure SAPe is estimated from the relationship shown in FIG. 24, based on the compression pressure Pc indicative of the real magnitude at the local maximum site of the pulse wave MW obtained at the monitor pressure PcHm from the living body 14 as the target to be measured. Although in FIG. 24 the estimation is made assuming the linear relationship, the non-linear relationship such as the exponential function may be assumed for estimation.

At S53 to S55, similarly to S22 to S24 of FIG. 19, the estimated diastolic arterial pressure DAPe and estimated systolic arterial pressure SAPe estimated are stored, and displayed on the display device 78. While the stop (off) operation by the blood pressure estimation start operation button 80 continues to be negative, the blood pressure estimation routine at S46 and subsequent steps are repeated and, if the stop (off) operation by the blood pressure estimation start operation button 80 goes affirmative, the blood pressure monitoring routine is brought to an end.

As described above, according to the electronic control device 170 of this embodiment, since Formula (6) i.e. a proper relationship expression of the living body 14 among the estimated notch arterial pressure DNAPe, the compression pressure, and the pulse wave propagation velocity is generated in the proper relationship generation portion 192, by using the real notch arterial pressure DNAP_{R} of the living body 14 as the person to be measured, the first keep pressure PcH1 and the second keep pressure PcH2 that are real compression pressures, and the pulse wave propagation velocities PWV1_{DN} and PWV2_{DN} based on the time differences Δt113_{DN} and Δt213_{DN} between the notch sites of the pulse waves obtained respectively under the first keep pressure PcH1 and the second keep pressure PcH2 that are real compression pressures, the blood pressure estimation portion 194 can easily estimate the estimated notch arterial pressure DNAPe of the living body 14, by applying to Formula (6) that is a proper relationship expression of the living body generated by the proper relationship generation portion 192 the real monitor pressure PcHm obtained at the low pressure section lower than the diastolic arterial pressure DAP of the living body 14 and the pulse wave propagation velocity PWV_{DN} based on the time difference between the notch sites of the pulse waves obtained under the real monitor pressure PcHm.

According to the electronic control device 170 of this embodiment, the propagation times (time differences Δt113_{DN} and Δt213_{DN}) between the notch sites of the pair of pulse waves obtained respectively for the plurality of keep pressures i.e. the first keep pressure PcH1 and the second keep pressure PcH2 are propagation times between the zero-crossing points from negative toward positive of the first derivative waveforms of the pulse waves. This facilitates obtainment of the propagation time between the notch sites of the pair of pulse waves, leading to enhanced estimation accuracy of the estimated notch arterial pressure DNAPe.

According to the electronic control device 170 of this embodiment, since the blood pressure estimation portion 194 includes the notch arterial pressure estimation portion 200 that estimates the estimated notch arterial pressure DNAPe of the living body 14 by successively applying to the proper relationship of Formula (6) the real monitor pressure PcHm in the low pressure section lower than the diastolic arterial pressure DAP of the living body 14 as the person to be measured and the real pulse wave propagation velocity PWVm_{DN} obtained under the monitor pressure PcHm, the blood pressure estimation portion 194 can easily estimate the estimated notch arterial pressure DNAPe of the living body 14.

According to the electronic control device 170 of this embodiment, the blood pressure estimation portion 194 includes: the diastolic arterial pressure estimation portion 196 that estimates the estimated diastolic arterial pressure DAPe by successively applying to the proper relationship of Formula (2) the real monitor pressure PcHm in the low pressure section lower than the diastolic arterial pressure DAP of the living body 14 as the person to be measured and the real pulse wave propagation velocity PWVm_{D} obtained under the monitor pressure PcHm; and the systolic arterial pressure estimation portion 198 that estimates the estimated systolic arterial pressure SAPe by generating a relationship (FIG. 24) between the estimated arterial pressure APe and the magnitude of the pulse wave at the monitor pressure PcHm section lower than the diastolic arterial pressure DAP, based on the estimated diastolic arterial pressure DAPe estimated by the diastolic arterial pressure estimation portion 196 and the estimated notch arterial pressure DNAPe estimated by the notch arterial pressure estimation portion 200, and applying to the relationship the real maximum value of the pulse wave successively obtained under the monitor pressure PcHm. This enables easy estimation of the estimated systolic arterial pressure SAPe of the person to be measured even in the case where the time difference between the local minimum sites of the pair of pulse waves successively obtained under the monitor pressure PcHm cannot be correctly found.

Although one embodiment of the present invention has hereinabove been described in detail with reference to the drawings, the present invention can be carried out in other modes without being limited to the embodiment.

Although for example, in the above blood pressure monitoring apparatus 10 both the estimated systolic arterial pressure SAPe and the estimated diastolic arterial pressure DAPe have been estimated, the configuration may be such that one of the estimated systolic arterial pressure SAPe and the estimated diastolic arterial pressure DAPe is estimated. In this case, for example, one of the regression lines of Formulae (1) and (3) stored in the linear relationship storage portion 82 is unnecessary, and one of the diastolic arterial pressure estimation portion 96 and the systolic arterial pressure estimation portion 98, etc. are unnecessary.

In the above embodiment, a plurality of pulse waves may be extracted for each of the first keep section keeping the first keep pressure PcH1, the second keep section keeping the second keep pressure PcH2, and the monitor pressure keep section keeping the monitor pressure PcHm, and a mean value of the time differences sampled from those plural pulse waves may be used.

Although in the first embodiment and the second embodiment, the cuff 12 has included the three inflatable bladders i.e. the upstream inflatable bladder 22, the intermediate inflatable bladder 24, and downstream inflatable bladder 26, it need only include at least two inflatable bladders.

Although in the first embodiment and the second embodiment the stepwise pressure lowering has been employed for the cuff 12, continuous gradual pressure lowering may be employed.

The above are mere embodiments, and although not exemplified one by one in addition thereto, the present invention can be carried out in modes variously altered or modified based on the knowledge of those skilled in the art without departing from the gist thereof.

### EXPLANATIONS OF LETTERS OR NUMERALS

10, 110: blood pressure monitoring apparatus
12: cuff
14: living body (person to be measured)
16: upper arm (site to be compressed)
18: artery
22: upstream inflatable bladder (inflatable bladder)
24: intermediate inflatable bladder (inflatable bladder)
26: downstream inflatable bladder (inflatable bladder)
82, 182: linear relationship storage portion
84, 184: blood pressure measurement portion
86, 186: compression pressure control portion
88, 188: pulse wave extraction portion
90, 190: pulse wave propagation velocity calculation portion
92, 192: proper relationship generation portion
94, 194: blood pressure estimation portion
96, 196: diastolic arterial pressure estimation portion (blood pressure estimation portion)
98, 198: systolic arterial pressure estimation portion (blood pressure estimation portion)
200: notch arterial pressure estimation portion

1. A blood pressure monitoring apparatus including a cuff wrapped around a site to be compressed of a person to be measured to compress an artery of the person to be measured, the cuff having a plurality of inflatable bladders forming independent air chambers juxtaposed across width, the blood pressure monitoring apparatus repeatedly estimating an estimated arterial pressure of the person to be measured, the blood pressure monitoring apparatus comprising:
a linear relationship storage portion storing previously stored linear relationships between a plurality of transmural pressures of the artery that are pressure differences between an arterial pressure within the artery and a plurality of compression pressures of the cuff, and squared values of pulse wave propagation velocities respectively detected under the plurality of compression pressures of the cuff in a low pressure section lower than a diastolic arterial pressure of a living body;
a blood pressure measurement portion measuring a real arterial pressure of the person to be measured, based on a pulse synchronous wave from the artery obtained in a pressure lowering process after compressing the site to be compressed of the person to be measured with a compression pressure higher than a systolic arterial pressure of the person to be measured;
a proper relationship generation portion applying, for the person to be measured, the real arterial pressure, real compression pressures in the low pressure section, and real pulse wave propagation velocities based on propagation time between the pulse waves obtained respectively under the real compression pressures, to thereby generate a proper relationship on the person to be measured among the real arterial pressures of the person to be measured, the real compression pressures, and the real pulse wave propagation velocities; and
a blood pressure estimation portion applying, for the person to be measured, the real compression pressures in the low pressure section and the real pulse wave propagation velocities obtained under the real compression pressures, to the proper relationship on the person to be measured, to thereby estimate the estimated arterial pressure.

2. The blood pressure monitoring apparatus of claim 1, wherein
the estimated arterial pressure estimated by the blood pressure estimation portion is an estimated diastolic arterial pressure DAPe of the person to be measured, and wherein
the linear relationship is a regression line expressed by Formula (1) below:
PWV²=s·(DAP−Pc)+i (1)
where PWV is the pulse wave propagation velocity of the living body, DAP is the diastolic arterial pressure of the living body, and Pc is the compression pressure on the living body, and
where s denotes a slope of the regression line and i denotes an intercept of the regression line.

3. The blood pressure monitoring apparatus of claim 2, wherein
the proper relationship on the person to be measured is expressed by Formula (2) below:
DAPe=PWV_{D}²/s_{D}−i_{D}/s_{D}+Pc (2)
where i_{D} and s_{D} are really measured calibration values, obtained respectively as solutions to unknowns i_{D} and s_{D} when: substituting, into two equations each expressed by Formula (1), a diastolic arterial pressure really measured on the person to be measured, as DAP; substituting thereinto different real compression pressures within the low pressure section, respectively, as Pc; and substituting thereinto real pulse wave propagation velocities based on propagation time between local minimum sites of pulse waves obtained respectively for the different real compression pressures, respectively, as PWV_{D}.

4. The blood pressure monitoring apparatus of claim 3, wherein
the propagation time between the local minimum sites of the pulse waves obtained respectively for the real compression pressures is propagation time between vertices occurring correspondingly to rising points of the pulse waves obtained respectively for the real compression pressures, in second derivative waveforms of the pulse waves obtained respectively for the real compression pressures.

5. The blood pressure monitoring apparatus of claim 3, wherein
the blood pressure estimation portion comprises a diastolic arterial pressure estimation portion estimating the estimated diastolic arterial pressure, by successively applying, for the person to be measured, real compression pressures in the low pressure section and the real pulse wave propagation velocities obtained under the real compression pressures, to the proper relationship of Formula (2).

6. The blood pressure monitoring apparatus of claim 1, wherein
the estimated arterial pressure estimated by the blood pressure estimation portion is an estimated systolic arterial pressure SAPe of the person to be measured, and wherein
the linear relationship is a regression line expressed by Formula (3) below:
PWV²=s·(SAP−Pc)+i (3)
where PWV is the pulse wave propagation velocity of the living body, SAP is the systolic arterial pressure of the living body, and Pc is the compression pressure on the living body, and
where s denotes a slope of the regression line and i denotes an intercept of the regression line.

7. The blood pressure monitoring apparatus of claim 6, wherein
the proper relationship on the person to be measured is expressed by Formula (4) below:
SAPe=PWV_{S}²/s_{S}−i_{S}/s_{S}+Pc (4)
where i_{S} and s_{S} are really measured calibration values, obtained as solutions to unknowns i and s when: substituting, into two equations each expressed by Formula (3), a systolic arterial pressure measured on the person to be measured, as SAP; substituting thereinto different real compression pressures within the low pressure section, respectively, as Pc; and substituting thereinto real pulse wave propagation velocities based on propagation time between local maximum sites of pulse waves obtained respectively for the different real compression pressures, respectively, as PWV_{S}.

8. The blood pressure monitoring apparatus of claim 7, wherein
the propagation time between local maximum sites of pulse waves obtained respectively for the real compression pressures is propagation time between local maximum points of pulse waves obtained respectively for the real compression pressures.

9. The blood pressure monitoring apparatus of claim 7, wherein
the blood pressure estimation portion comprises a systolic arterial pressure estimation portion estimating the estimated systolic arterial pressure, by successively applying, for the person to be measured, real compression pressures in the low pressure section and the real pulse wave propagation velocities obtained under the real compression pressures, to the proper relationship of Formula (4).

10. The blood pressure monitoring apparatus of claim 1, wherein
the estimated arterial pressure estimated by the blood pressure estimation portion is an estimated notch arterial pressure DNAPe of the person to be measured that is a compression pressure upon occurrence of notch sites locally formed posterior to local maximum sites of pulse waves obtained respectively for the real compression pressures, and wherein
the linear relationship is a regression line expressed by Formula (5) below:
PWV²=s·(DNAP−Pc)+i (5)
where PWV is the pulse wave propagation velocity of the living body, DNAP is the notch arterial pressure of the living body, and Pc is the compression pressure on the living body, and
where s denotes a slope of the regression line and i denotes an intercept of the regression line.

11. The blood pressure monitoring apparatus of claim 10, wherein
the proper relationship on the person to be measured is expressed by Formula (6) below:
DNAPe=PWV_{DN}²/s_{DN}−i_{DN}/s_{DN}+Pc (6)
where i_{DN} and s_{DN} are really measured calibration values, obtained as solutions to unknowns i and s when: substituting, into two equations each expressed by Formula (5), a notch arterial pressure really measured on the person to be measured, as DNAP; substituting thereinto different real compression pressures within the low pressure section, respectively, as Pc; and substituting thereinto real pulse wave propagation velocities based on propagation time between notch sites of pulse waves obtained respectively for the different real compression pressures, respectively, as PWV_{DN}.

12. The blood pressure monitoring apparatus of claim 11, wherein
the propagation time between notch sites of the pulse waves obtained respectively for the real compression pressures is propagation time between vertices occurring posterior to time points corresponding to local maximum sites of pulse waves obtained respectively for the real compression pressures, in second derivative waveforms of the pulse waves obtained respectively for the real compression pressures.

13. The blood pressure monitoring apparatus of claim 11, wherein
the blood pressure estimation portion comprises a notch arterial pressure estimation portion estimating the estimated notch arterial pressure, by successively applying, for the person to be measured, real compression pressures in the low pressure section and the real pulse wave propagation velocities obtained under the real compression pressures, to the proper relationship of Formula (6).

14. The blood pressure monitoring apparatus of claim 13, wherein
the blood pressure estimation portion comprises:
a diastolic arterial pressure estimation portion estimating an estimated diastolic arterial pressure of the person to be measured, by successively applying, for the person to be measured, real compression pressures in the low pressure section and real pulse wave propagation velocities obtained under the real compression pressures, to a proper relationship among the diastolic arterial pressures really measured on the person to be measured, the real compression pressures in the low pressure section, and the real pulse wave propagation velocities in the low pressure section; and
a systolic arterial pressure estimation portion estimating an estimated systolic arterial pressure, by generating a relationship between magnitudes of pulse waves in the low pressure section and the estimated arterial pressures, based on the estimated diastolic arterial pressure estimated by the diastolic arterial pressure estimation portion and the estimated notch arterial pressure estimated by the notch arterial pressure estimation portion, and applying real maximum values of pulse waves successively obtained, to the relationship.

15. The blood pressure monitoring apparatus of claim 1, comprising:
a compression pressure control portion stepwise lowering a plurality of compression pressures within the low pressure section so as to form a plurality of sections temporarily keeping the plurality of compression pressures at constant values in the low pressure section;
a pulse wave extraction portion extracting pulse waves that are pressure oscillations occurring in synchronization with pulses within each of the plurality of inflatable bladders under compression pressures in the plurality of sections; and
a pulse wave propagation velocity calculation portion calculating the pulse wave propagation velocity, based on time difference between pulse waves obtained in each of the plurality of sections and length between the plurality of inflatable bladders.

16. The blood pressure monitoring apparatus of claim 1, wherein
the cuff is wrapped around a site to be compressed of a living body and has an upstream inflatable bladder, an intermediate inflatable bladder, and a downstream inflatable bladder independent of each other and juxtaposed across width, each compressing the site to be compressed of the living body, and wherein
the artery within the site to be compressed is compressed with an equal compression pressure by the upstream inflatable bladder, the intermediate inflatable bladder, and the downstream inflatable bladder.
